(19) United States
(12) Patent Application Publication
(10) Pub. No.: US 2025/0250633 A1
(43) Pub. Date: Aug. 7, 2025

(54) DIFFERENTIAL AMPLIFICATION OF CIRCULAR POLYNUCLEOTIDES

(71) Applicant: Singular Genomics Systems, Inc., San Diego, CA (US)

(72) Inventors: Eli N. Glezer, Del Mar, CA (US); Andrew Pawlowski, San Diego, CA (US); Michael Lawson, Pasadena, CA (US); Yuji Ishitsuka, San Diego, CA (US); Daan Witters, San Diego, CA (US)

(21) Appl. No.: 19/042,521

(22) Filed: Jan. 31, 2025

Related U.S. Application Data
(60) Provisional application No. 63/549,152, filed on Feb. 2, 2024.
(60) Provisional application No. 63/652,914, filed on May 29, 2024.

(51) Int. Cl. C12Q 1/6876; C12Q 1/682; C12Q 1/6841

(52) U.S. Cl. CPC C12Q 1/6876; C12Q 1/682; C12Q 1/6841; C12Q 2600/16

(57) ABSTRACT

Disclosed herein, inter alia, are compositions and methods for amplifying a dynamic range of nucleic acid molecules.

### CROSS-REFERENCE TO RELATED APPLICATIONS

This application claims the benefit of U.S. Provisional Application No. 63/549,152, filed Feb. 2, 2024 and U.S. Provisional Application No. 63/652,914, filed May 29, 2024; each of which are incorporated herein by reference in their entirety and for all purposes.

### BACKGROUND

Simultaneous detection of different nucleic acid targets in situ, particularly when there are significant disparities in the expression levels of those different targets, has historically posed several challenges. For example, in cases where some targets are expressed at much higher quantities than others, there is a risk of signal overlap and/or oversaturation when using fluorescent based detection. High-abundance targets can produce strong signals that overshadow or mask the detection of low-abundance targets. This wide dynamic range imbalance makes it difficult to accurately detect and quantify multiple targets within the same sample. Disclosed herein, inter alia, are solutions to these and other problems in the art.

### BRIEF SUMMARY

In an aspect is provided a method for differentially amplifying polynucleotides in a cell or tissue. In embodiments, the method includes (i) contacting a cell or tissue with a plurality of polymerases and deoxynucleotide triphosphates (dNTPs) and (ii) amplifying a first circular polynucleotide including a first sequence to generate an amplification product including a first number of copies of the first sequence and amplifying a second circular polynucleotide including a second sequence to generate an amplification product including a second number of copies of the second sequence; wherein the first number is detectably less than the second number, wherein the first polynucleotide is hybridized to a first nucleic acid molecule covalently attached to a specific binding agent, wherein the first circular polynucleotide includes a retarding agent; and the second polynucleotide is hybridized to a second nucleic acid molecule. In embodiments, the specific binding is a protein-specific binding agent.

### BRIEF DESCRIPTION OF THE DRAWINGS

FIGS. 1A-1F. Illustrations of the components useful for the methods described herein for the detection of protein targets in situ. FIG. 1A shows a first probe (1) oligonucleotide (e.g., a single stranded-polynucleotide described herein), including a first 5′ arm region (A′), a first linker region (2), and a first 3′ arm region (B′). FIG. 1B illustrates an antibody-oligonucleotide conjugate (Ab-O; 3), which includes a protein-specific binding molecule (e.g., an antibody; 4), attached to an oligonucleotide (5) (e.g., a first nucleic acid molecule described herein). The oligonucleotide includes, from 5′ to 3′, a first hybridization sequence (A) which is complementary to the first 5′ arm region (A′) of the first probe illustrated in FIG. 1A, a target-specific sequence (6) (e.g., a barcode sequence, UMI, also referred to as a unique molecular identifying sequence, or an identifying nucleotide associated with the identity of the specific binding reagent), and a second hybridization sequence (B) which is complementary to the 3′ arm region (B′) of the first probe illustrated in FIG. 1A. The 3′ ends of the oligonucleotide may include modifications (e.g., dideoxynucleotide or a reversibly-terminated nucleotide) to prevent 3′ extension and potential displacement. When applied to a biological sample (e.g., a cell or tissue), the Ab-O binds specifically to its target (e.g., a protein; 7), thus forming a target-probe complex (e.g., 8, as illustrated in FIG. 1C). FIGS. 1C-1D illustrate how the first probe anneals to the oligonucleotide of the target probe complex, and how once annealed, the 3′ end of the PLP (B′) may be extended to generate a complement of the target-specific sequence (9), and a ligase then joins the adjacent ends to form a circular polynucleotide (10). In alternative embodiments, the target specific sequence is absent (not shown), the 5′ arm region (A′) and the 3′ arm region (B′) anneal to adjacent sequences, and ligation of the two regions occurs absent an extension step to form a circular polynucleotide. In embodiments, the first probe includes a barcode sequence, UMI, also referred to as a unique molecular identifying sequence, or an identifying nucleotide associated with the identity of the specific binding reagent. FIG. 1E illustrates how, once formed, the circular polynucleotide may be amplified via an amplification primer (11) and a polymerase (12), which generates an amplification product as illustrated in FIG. 1F. Alternatively, the oligonucleotide of the Ab-O conjugate may serve as the amplification primer for a rolling circle amplification process. FIG. 1F illustrates how the amplification product contains multiple copies of the target-specific sequence (6), which can then be detected (e.g., via binding of labeled probes or via sequencing as described herein). As illustrated in FIG. 1F, the amplification product contains 10 copies of the target-specific sequence.

FIGS. 2A-2D. Illustrations of the components useful for the methods described herein for the detection of nucleic acid targets in situ. FIG. 2A shows a probe oligonucleotide (13) (e.g., a single stranded-polynucleotide described herein), including a 5′ arm region (C′), a linker region (14), and a 3′ arm region (D′). FIG. 2B provides an illustrative example of a nucleic acid target molecule (15) that can be detected in situ via the probe illustrated in FIG. 2A. The nucleic acid target molecule includes, from 5′ to 3′, a first hybridization sequence (C) which is complementary to the 5′ arm region (C′) of the probe illustrated in FIG. 2A, a target sequence (16), and a second hybridization sequence (D) which is complementary to the second 3′ arm region (D′) of the probe in FIG. 2A. FIGS. 2C-2D illustrate how the probe anneals to the nucleic acid target molecule illustrated in FIG. 2B, and how, once annealed, the 3′ end of the PLP (D′) may be extended to generate a complement of the target sequence (17), and a ligase then joins the adjacent ends to form a circular polynucleotide (18) which may be amplified via an amplification primer and a polymerase, to generate a second amplification product. The second amplification product contains multiple copies of the target-specific sequence, which can then be detected using the methods described herein. Alternatively, the circular polynucleotide may be formed by binding the 5′ arm region (A′) and the 3′ arm region (B′) anneal to adjacent sequences of the target molecule, and ligation of the two regions occurs absent an extension step. In embodiments, the first probe includes a barcode sequence, UMI, also referred to as a unique molecular identifying sequence, or an identifying nucleotide associated with the identity of the specific binding reagent. In embodiments, the hybridization sequences, or the complements thereof, may be detected to identify the target of interest.

FIGS. 3A-3B. FIGS. 3A-3B illustrate how the probe illustrated in FIG. 1A (e.g., a single stranded-polynucleotide described herein) can be modified with a retarding agent (19, 20) to slow down the generation of the first amplification product illustrated in FIG. 1F. FIG. 3A illustrates an embodiment where the retarding agent includes a modified nucleotide (19) within the first PLP linker region. FIG. 3B illustrates an embodiment where the retarding agent includes a double-stranded region, illustrated as a hairpin loop (20) within the linker region.

FIGS. 4A-4B. FIG. 4A illustrates how amplification of a circular polynucleotide described herein including a retarding agent (top left) is slowed down relative to a circular polynucleotide that lacks a retarding agent (top right). Due presence the retarding agent, concurrent amplification provides the amplification product of the affected circularized polynucleotide (bottom left) has a lower number of copies of the target sequence (21) (e.g., 4 copies in this particular example) relative to the number of copies of the target sequence (e.g., 8 copies in this particular example) in the amplification product of the nonaffected probe (bottom right). In this particular illustrative example, the retarding agent is a modified nucleotide (19). FIG. 4B illustrates a similar concept as that of FIG. 4A, albeit in this particular example the retarding agent is a hairpin loop (20).

FIGS. 5A-5B. FIG. 5A illustrates additional embodiments of the circularized polynucleotide. The circularized polynucleotide may include one or more primer binding sequences (e.g., an amplification and/or sequencing primer binding sequence). In embodiments, the amplification primer binding sequence is located within the 3′ arm (A′; top left panel), the 5′ arm (B′; top right panel), and/or within the linker region (bottom left and right panels) of the circularized polynucleotide. In this particular example, each circularized probe includes a retarding agent (19), and demonstrates how the amplification primer binding sites can be placed at variable distances from the retarding agent. For example, an amplification primer binding site can be placed upstream or downstream of the retarding agent, for example, in close proximity to the 3′ end (22) or 5′ end (23) of the retarding agent, conferring greater control over the amplification rate of a circularized polynucleotide. FIG. 5B illustrates a similar concept as that of FIG. 5A, albeit in this particular example the retarding agent is a hairpin loop (20).

FIG. 6 illustrates additional embodiments where the length of the hairpin retarding agent is varied (24-27) as another means of controlling the rate of amplification.

FIG. 7 illustrates an embodiment for the simultaneous detection of proteins and nucleic acids within a cell. In this example, a first circularized polynucleotide (28), which includes a retarding agent (e.g., a hairpin) within its linker region, is used for the detection of a target protein. Also present within the same cell is a second circularized polynucleotide Φ29) lacking a retarding agent, which used for detecting a target nucleic acid. Due to the presence of the retarding agent, the first circularized polynucleotide undergoes amplification at a reduced rate, resulting in the generation of an amplification product (30) containing fewer copies of its target-specific sequence (32). The second circularized polynucleotide, on the other hand, amplifies more efficiently, resulting in the generation of an amplification product (31) containing fewer copies of its target-specific sequence (33).

FIGS. 8A-8B. FIGS. 8A-8B provide experimental evidence of reduced amplification. We designed probes containing one or two consecutive 2′OMe modified bases and subjected them to intramolecular ligation in vitro to generate single stranded DNA circles. The circular probes labeled 1 mC (or mC) corresponds to a probe including a 2′OMe modified cytosine; 1 mA (or mA) corresponds to a probe inclusive of a 2′OMe modified adenine; 1 mG (or mG) corresponds to a probe inclusive of a 2′OMe modified guanine; 1 mU (or mU) corresponds to a probe inclusive of a 2′OMe modified uracil; 2 mC corresponds to a probe inclusive of two consecutive 2′OMe modified cytosines; 2 mA corresponds to a probe inclusive of two consecutive 2′OMe modified adenines; and NTC is the non-template control probe used for the assay. The efficiency of rolling circle amplification on each padlock probe design was assayed by continuous monitoring of phi29 amplicon products using SYTO™ 9 fluorescent dye over a 12 hour period compared to a control probe consisting of natural bases. FIG. 8A shows the detected fluorescence over time, and shows presence of a retarding agent (e.g., 1 mC, 1 mA, 1 mG, and 1 mU) slows amplification relative to the control probe. Additionally, probes including two consecutive retarding agents (e.g., 2 mA) significantly impacts amplification, measurably indistinct from the NTC. The 2′OMe probe designs were then evaluated using fluorescence in situ hybridization assays to evaluate amplicon signal intensity and antigen labeling efficiency following 3 or 16 hours of in situ RCA (FIG. 8B). The white scale bar shown in each image corresponds to 200 μm. The relative abundance of the desired target may factor into the choice of the identity and quantity of the retarding agent.

FIGS. 9A-9B. Shown in FIG. 9A is the fluorescent in situ hybridization (FISH) and quantification of amplification products for various time points when detecting CD3e proteins in tonsil. Antibody-oligo (Ab-O) conjugates specific for CD3a proteins were incubated using standard staining conditions. Circularizable polynucleotides (e.g., single-stranded polynucleotides described herein) targeting the oligonucleotide sequences of the Ab-O were incubated and amplified. An unmodified circular polynucleotide (referred to as the standard probe) amplified for 15 minutes and the detection products was quantified. Amplification products for a biotin probe, 1 mU probe, 1 mG probe and 2 mC probe were detected at 1 hour, 2 hour, and 4 hour time intervals. Additionally, we confirm that biotinylated padlock effectively reduces the rate of RCA without hindering cell detection or cell morphology as captured in the figures presented in FIG. 9B. The scale bar shown in each image on the top row of FIG. 9B represents 200 μm. The bottom row of FIG. 9B represents the magnified view of the enclosed region of the corresponding image in the top row. The scale bar shown in the bottom row of FIG. 9B represents 30 μm.

FIG. 10 provides an illustration of the sequential collection of information to inform on the structure of a cell and/or tissue. Spectrally distinct dyes are used in the first set, and optionally reused in subsequent sets. For example, the first set includes Alexa Fluor® 532 (emission: 532 nm), Alexa Fluor® 594 (emission: 594 nm), Alexa Fluor® 647 (emission: 647 nm), and Alexa Fluor® 680 (emission: 680 nm) to illuminate the Golgi Apparatus, endoplasmic reticulum, actin, lysosomes, and specific cell surface receptors of a cell. Following cleavage and removal of the fluorophores, the second set of targeting molecules are incubated with the sample cell. The second set can then illuminate the nucleus, nucleoli, mitochondria, nuclear envelop, cell surface receptors, and plasma membrane. The sequential addition of cell paints can continue for N cycles providing additional information about the cell. The resulting images may be computationally processed and overlaid to provide a composite image of the cell and/or tissue.

### DETAILED DESCRIPTION

Described herein are novel methods for differentially amplifying polynucleotides, optionally within a cell or tissue environment (i.e., in situ).

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the term “blocking oligonucleotide” refers to an oligonucleotide hybridized to a polynucleotide (e.g., a circular polynucleotide described herein) or a portion of a polynucleotide described herein. The blocking oligonucleotide serves to stall or retard a polymerase from amplifying the polynucleotide hybridized to the blocking oligonucleotide described herein.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(O), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently KS P C

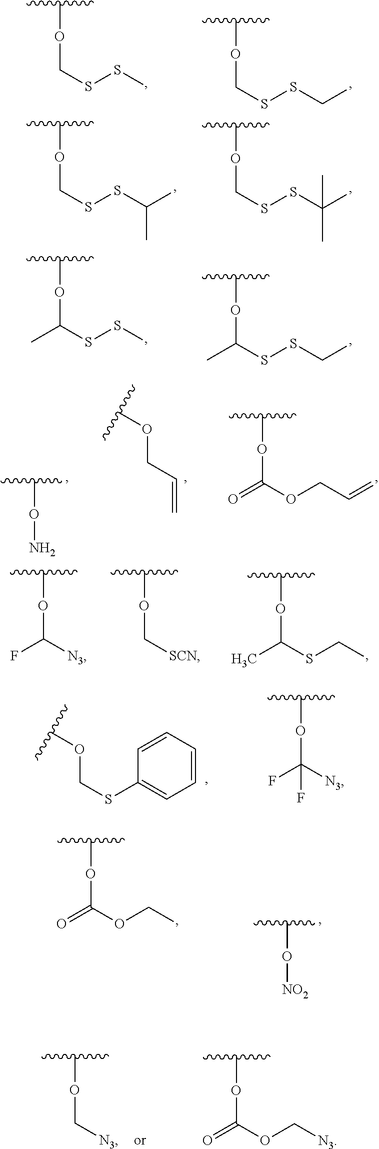

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

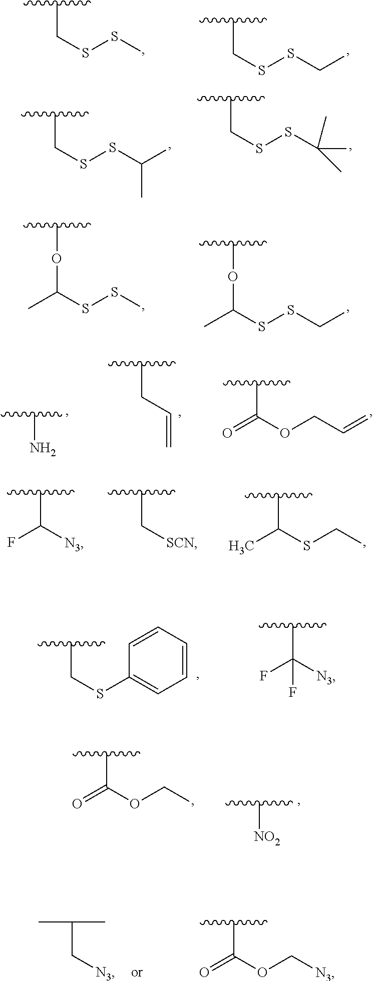

wherein the 3′ oxygen of the nucleotide is not shown in the formulae above. The term “allyl” as described herein refers to an unsubstituted methylene attached to a vinyl group (i.e., —CH═CH₂). In embodiments, the reversible terminator moiety is

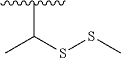 [SMILES: *C(C)SSC]

as described in U.S. Pat. No. 10,738,072, which is incorporated herein by reference for all purposes. For example, a nucleotide including a reversible terminator moiety may be represented by the formula:

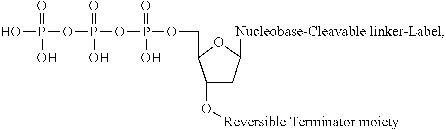

where the nucleobase is adenine or adenine analogue, thymine or thymine analogue, guanine or guanine analogue, or cytosine or cytosine analogue.

In some embodiments, a nucleic acid (e.g., a probe or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcodes are selected to form a known set of barcodes, e.g., the set of barcodes may be distinguished by a particular Hamming distance. In embodiments, each barcode sequence is unique within the known set of barcodes. In embodiments, each barcode sequence is associated with a particular oligonucleotide probe.

In embodiments, a nucleic acid (e.g., an adapter or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. Typically, a DNA polymerase adds nucleotides to the 3′-end of a DNA strand, one nucleotide at a time. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g., apoptosis or necrosis) or cell shedding, releasing polynucleotides into surrounding body fluids or into circulation. Accordingly, cell-free polynucleotides may be isolated from a non-cellular fraction of blood (e.g., serum or plasma), from other bodily fluids (e.g., urine), or from non-cellular fractions of other types of samples.

As used herein, the terms “specific”, “specifically”, “specificity”, or the like of a compound refers to the compound's ability to cause a particular action, such as binding, to a particular molecular target with minimal or no action to other proteins in the cell.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. In embodiments, the KD (equilibrium dissociation constant) between two specific binding molecules is less than 10⁻⁶ M, less than 10⁻⁷ M, less than 10⁻⁸ M, less than 10⁻⁹ M, less than 10⁻¹⁰ M, less than 10⁻¹¹ M, or less than about 10⁻¹² M or less.

As used herein, the term “specific binding agent” refers to an agent that binds specifically to a particular biomolecule (e.g., carbohydrate, cell surface receptor, protein, nucleic acid, or lipid molecule). Examples of a specific binding reagent include, but are not limited to, an antibody or target-specific oligonucleotide.

As used herein, the term “protein-specific binding agent” refers to an agent that specifically interacts and binds to proteins. Examples of protein-binding agents include but are not limited to antibodies.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can include natural or synthetic polymers.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell.

The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, the flow cell includes glass (e.g., borosilicate glass, soda lime glass, etc.), fused silica (quartz), polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), polydimethylsiloxane (PDMS), etc.), or polyetherimide (PEI), or any combination thereof. In embodiments, a flow cell includes inlet and outlet ports and a flow channel extending there between. Typically, the flow cell shape includes flat surfaces that can reside within the focal depth of the FOV of the microscope imaging system.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction includes a suitable thermal stable polymerase. Thermal stable polymerases are known and are stable for prolonged periods of time, at temperature greater than 80° C. when compared to common polymerases found in most mammals. In certain embodiments the term “amplification,” “amplified” or “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays including colonies (or “clusters”) of immobilized nucleic acid molecule.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin; Barany, Proc. Natl. Acad. Sci. USA 88:188-93 (1991); Bi and Sambrook, Nucl. Acids Res. 25:2924-2951 (1997); Zirvi et al., Nucl. Acid Res. 27: e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

As used herein, the terms “cluster” and “colony” are used interchangeably to refer to a discrete site that includes a plurality of polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

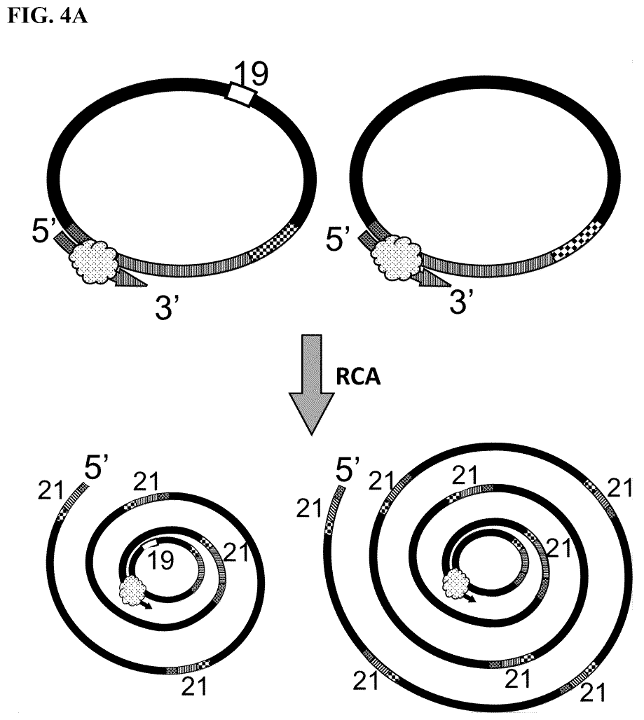

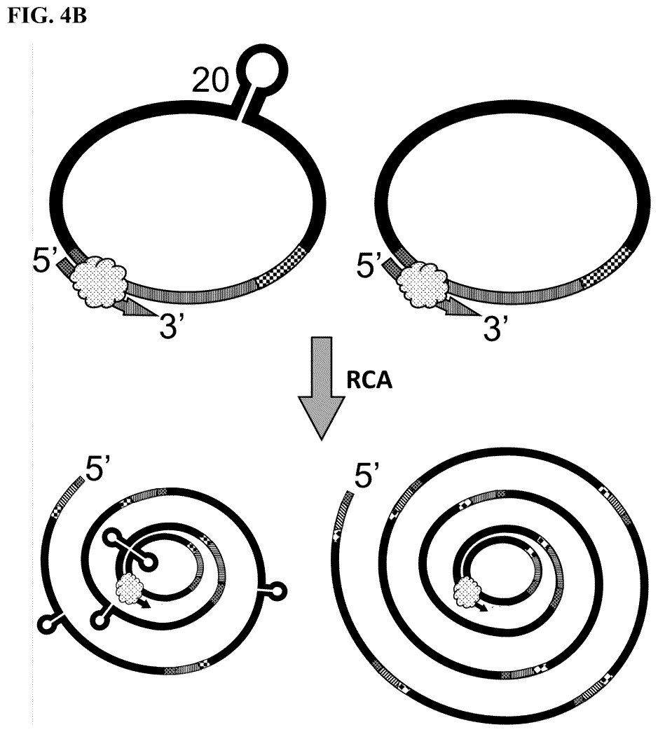

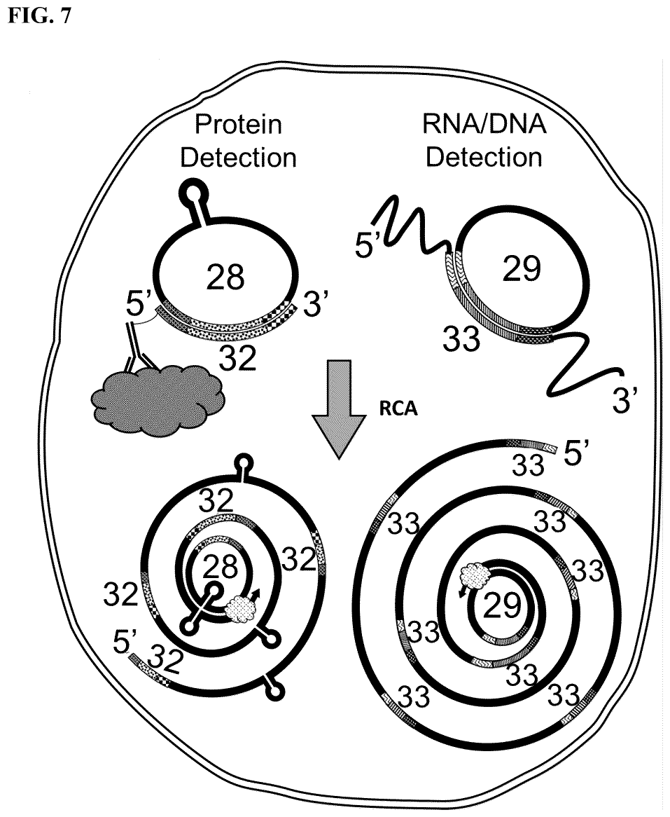

As used herein, the methods described herein are for “differentially amplifying polynucleotides.” As used herein, “differentially amplifying polynucleotides” refers to an amplification method including amplifying a first circular polynucleotide and amplifying a second circular polynucleotide, wherein the first circular polynucleotide includes a retarding agent as described herein that reduces the amplification of the first polynucleotide compared to the amplification of the second polynucleotide. Examples of differentially amplifying polynucleotides are provided in, but are not limited to, FIGS. 4A, 4B, and 7.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

The term “organelle” as used herein refers to an entity of cell associated with a particular function. In embodiments, an organelle refers to a specialized subunit within a cell that has a specific function, and is usually separately enclosed within its own lipid bilayer. Examples of organelles include the nucleus, mitochondria, endoplasmic reticulum, Golgi apparatus, lysosomes, and chloroplasts (in plant cells). Although most organelles are functional units within cells, some organelles function extend outside of cells, such as cilia, flagellum, archaellum, and the trichocyst. In embodiments, the organelle is a membrane bound organelle. In embodiments, the organelle is a non-membrane bound organelle. Non-membrane bounded organelles, also called biomolecular complexes, are assemblies of macromolecules such as the ribosome, the spliceosome, the proteasome, the nucleosome, and the centriole. Commonly detected organelles includes the nucleus, which is often visualized using dyes such as DAPI, Hoechst, and SYTO Green, mitochondria are with MitoTracker™ dyes and Rhodamine 123, endoplasmic reticulum (ER) utilizing dyes like ER-Tracker® Green/Red or DiOC6, the Golgi apparatus is stained with BODIPY™ FL C5-Ceramide and NBD C6-Ceramide, lysosomes are typically stained using LysoTracker™ dyes and Acridine Orange, and peroxisomes may be stained with Peroxisome-Tracker® Red and Peroxy Green dyes. Although not membrane-bound, ribosomes may detected using antibodies such as anti-RPL10 or anti-RPS6. Additionally, the cytoskeleton, specifically actin filaments, is frequently stained to study cell shape with Phalloidin conjugates and Alexa Fluor® Phalloidin being widely used. In embodiments, the organelle is a biomolecular complex including a plurality of subunits. In embodiments, the organelle is a macromolecule. In embodiments, the organelle is a eukaryotic organelle. In embodiments, the organelle is the cell membrane, the endoplasmic reticulum, a flagellum, a Golgi apparatus, a mitochondria, the nucleus, a vacuole. In embodiments, the organelle is a lysosome. In embodiments, the organelle is the nucleolus.

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid. A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate reactive group 1 | Bioconjugate reactive group 2 | |
|---|---|---|
| (e.g., electrophilic bioconjugate | (e.g., nucleophilic bioconjugate | Resulting Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or strepavidin to form a avidin-biotin complex or streptavidin-biotin complex.

An “antibody” (Ab) is a protein that binds specifically to a particular substance, known as an “antigen” (Ag). An “antibody” or “antigen-binding fragment” is an immunoglobulin that binds a specific “epitope.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)2, CDRs, VL, VH, and any other portion of an antibody which is capable of specifically binding to an antigen. Antibodies used herein are immunospecific for, and therefore specifically and selectively bind to, for example, proteins either detected (e.g., biological targets of interest) or used for detection (e.g., probes containing oligonucleotide barcodes) in the methods and devices as described herein.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, “biological activity” may include the in vivo activities of a compound or physiological responses that result upon in vivo administration of a compound, composition or other mixture. Biological activity, thus, may encompass therapeutic effects and pharmaceutical activity of such compounds, compositions and mixtures. Biological activities may be observed in vitro systems designed to test or use such activities.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ system), Jllumina™ (e.g., HiSeq™, MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH4)2SO4)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a glass slide. In embodiments, a tissue section, such as a formalin-fixed paraffin-embedded (FFPE) tissue section, is a biological specimen that has been preserved and prepared for microscopic examination and analysis. The FFPE preservation process involves fixing the tissue in formalin to prevent decay and embedding it in paraffin wax, which facilitates thin slicing and mounting on slides for histological examination. For example, embedding in paraffin wax not only supports the tissue for sectioning but also allows for long-term storage at room temperature without significant degradation of tissue morphology or molecular integrity. Tissue sections may be obtained from a piece of an embedded tissue, wherein the embedded tissue block is sliced to the desired thickness to provide a tissue section (e.g., 4-8 micrometers).

The term “retardant agent” or “retarding agent” refers to a complex, agent (e.g., a modified nucleotide), or molecule that is capable of slowing extension of a primer bound to a polynucleotide. In embodiments, a retarding agent is an element introduced into a reaction to deliberately and measurably decrease the rate DNA or RNA amplification. A retarding agent may act by inhibiting or altering the activity of enzymes involved in nucleic acid processes. For example, in DNA amplification, a retarding agent may reduce the efficiency or speed of DNA polymerase. A retarding agent can be designed to interact specifically with certain nucleotide sequences or structures within a nucleic acid molecule, thereby slowing down nucleotide incorporation processes. Retarding agents can take various forms, including modified nucleotides that are incorporated into a DNA or RNA strand but impede further extension by polymerases. In embodiments, the retarding moiety is a double-stranded polynucleotide (e.g., a hairpin). In embodiments, a retardant moiety increases the halftime of a further nucleotide extension to a level that is about or at least about 2-fold higher, 5-fold higher, 10-fold higher, 15-fold higher, 20-fold higher, 25-fold higher, 30-fold higher, or more, as compared to a control under conditions of an extension reaction.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect is provided a cell or tissue including a first amplification product and a second amplification product, wherein a first amplification product includes a first number of copies of a first sequence and the second amplification product includes a second number of copies of a second sequence; wherein the first number is detectably less than the second number. In embodiments, the cell or tissue is affixed (i.e., immobilized) to a solid support described herein.

In embodiments, the cell is an isolated single cell. In embodiments, the cell is a prokaryotic cell. In embodiments, the cell is a eukaryotic cell. In embodiments, the cell is a bacterial cell (e.g., a bacterial cell or bacterial spore), a fungal cell (e.g., a fungal spore), a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, ThO T cell, cytotoxic T cell); B cell, pre-B cell; monocytes; dendritic cell; neutrophils; or a macrophage. In embodiments, the cell is a stem cell, an immune cell, a cancer cell (e.g., a circulating tumor cell or cancer stem cell), a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell. In embodiments, the cell is a circulating tumor cell or cancer stem cell.

In embodiments, the cell is a prokaryotic cell. In embodiments, the cell is a bacterial cell. In embodiments, the bacterial cell is a Bacteroides, Clostridium, Faecalibacterium, Eubacterium, Ruminococcus, Peptococcus, Peptostreptococcus, or Bifidobacterium cell. In embodiments, the bacterial cell is a Bacteroides fragilis, Bacteroides melaninogenicus, Bacteroides oralis, Enterococcus faecalis, Escherichia coli, Enterobacter sp., Klebsiella sp., Bifidobacterium bifidum, Staphylococcus aureus, Lactobacillus, Clostridium perfringens, Proteus mirabilis, Clostridium tetani, Clostridium septicum, Pseudomonas aeruginosa, Salmonella enterica, Faecalibacterium prausnitzii, Peptostreptococcus sp., or Peptococcus sp. cell. In embodiments, the cell is a fungal cell. In embodiments, the fungal cell is a Candida, Saccharomyces, Aspergillus, Penicillium, Rhodotorula, Trametes, Pleospora, Sclerotinia, Bullera, or a Galactomyces cell.

In embodiments, the cell is a viral-host cell. A “viral-host cell” is used in accordance with its ordinary meaning in virology and refers to a cell that is infected with a viral genome (e.g., viral DNA or viral RNA). The cell, prior to infection with a viral genome, can be any cell that is susceptible to viral entry. In embodiments, the viral-host cell is a lytic viral-host cell. In embodiments, the viral-host cell is capable of producing viral protein. In embodiments, the viral-host cell is a lysogenic viral-host cell. In embodiments, the cell is a viral-host cell including a viral nucleic acid sequence, wherein the viral nucleic acid sequence is from a Hepadnaviridae, Adenoviridae, Herpesviridae, Poxviridae, Parvoviridae, Reoviridae, Coronaviridae, Retroviridae virus.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the cell is a neuronal cell, an endothelial cell, epithelial cell, germ cell, plasma cell, a muscle cell, peripheral blood mononuclear cell (PBMC), a myocardial cell, or a retina cell. In embodiments, the cell is a suspension cell (e.g., a cell free-floating in the culture medium, such a lymphoblast or hepatocyte). In embodiments, the cell is a glial cell (e.g., astrocyte, radial glia), pericyte, or stem cell (e.g., a neural stem cell). In embodiments, the cell is a neuronal cell. In embodiments, the cell is an endothelial cell. In embodiments, the cell is an epithelial cell. In embodiments, the cell is a germ cell. In embodiments, the cell is a plasma cell. In embodiments, the cell is a muscle cell. In embodiments, the cell is a peripheral blood mononuclear cell (PBMC). In embodiments, the cell is a myocardial cell. In embodiments, the cell is a retina cell. In embodiments, the cell is a lymphoblast. In embodiments, the cell is a hepatocyte. In embodiments, the cell is a glial cell. In embodiments, the cell is an astrocyte. In embodiments, the cell is a radial glia. In embodiments, the cell is a pericyte. In embodiments, the cell is a stem cell. In embodiments, the cell is a neural stem cell.

In embodiments, the cell is bound to a known antigen. In embodiments, the cell is a cell that selectively binds to a desired target, wherein the target is an antibody, or antigen binding fragment, an aptamer, affimer, non-immunoglobulin scaffold, small molecule, or genetic modifying agent. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a T cell, an NK cell, or a B cell.

In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell.

In embodiments, the cell is a cancer cell. In embodiments, the cancer is lung cancer, colorectal cancer, skin cancer, colon cancer, pancreatic cancer, breast cancer, cervical cancer, lymphoma, leukemia, or a cancer associated with aberrant K-Ras, aberrant APC, aberrant Smad4, aberrant p53, or aberrant TGFβ. In embodiments, the cancer cell includes a ERBB2, KRAS, TP53, PIK3CA, or FGFR2 gene. In embodiments, the cancer cell includes a HER2 gene. In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma.

In embodiments, the cell is a neuronal cell, an endothelial cell, epithelial cell, germ cell, plasma cell, a muscle cell, peripheral blood mononuclear cell (PBMC), a myocardial cell, cancer cell, or a retina cell.

In embodiments, the tissue incudes liver tissue, kidney tissue, bone tissue, lung tissue, thymus tissue, adrenal tissue, skin tissue, bladder tissue, colon tissue, spleen tissue, or brain tissue.

In embodiments, the tissue is a tissue section. In embodiments, the tissue section includes a tissue or a cell (e.g., plurality of cells such as blood cells). In embodiments, the tissue section includes one or more cells. In embodiments, the thickness of the tissue section is about 1 μm to about 20 μm. In embodiments, the thickness of the tissue section is about 5 μm to about 12 μm. In embodiments, the thickness of the tissue section is about 8 μm to about 15 μm. In embodiments, the thickness of the tissue section is about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, or about 15 μm.

In certain embodiments, tissue sections are tumor tissue samples. Tumor samples may contain only tumor cells, or they may contain both tumor cells and non-tumor cells. In particular embodiments, a tissue section includes only non-tumor cells. In particular embodiments, the tumor is a solid tumor. In particular embodiments, the tissue section is obtained from or includes an adrenal cortical cancer, anal cancer, aplastic anemia, bile duct cancer, bladder cancer, bone cancer, bone metastasis, brain tumor, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, head or neck cancer, Kaposi sarcoma, renal cell carcinoma, laryngeal and hypopharyngeal cancer, liver cancer, non-small cell lung cancer, small cell lung cancer, lung carcinoid tumor, lymphoma of the skin, malignant mesothelioma, myelodysplasia syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal or squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment, is a tissue section obtained from a subject diagnosed with or suspected of having any of these tumors or cancers.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation. In embodiments, the cell is permeabilized and immobilized to a solid support surface. In embodiments, the cell is permeabilized and immobilized to an array (i.e., to discrete locations arranged in an array). In embodiments, the cell is immobilized to a solid support surface. In embodiments, the tissue is permeabilized and immobilized to a solid support surface. In embodiments, the tissue is permeabilized and immobilized to an array (i.e., to discrete locations arranged in an array). In embodiments, the tissue is immobilized to a solid support surface.

In embodiments, the first amplification product is formed by amplifying a first circular polynucleotide including a first sequence to generate the first amplification product including a first number of copies of the first sequence. In embodiments the second amplification product is formed by and amplifying a second circular polynucleotide including a second sequence to generate the second amplification product including a second number of copies of the second sequence. In embodiments, the first circular polynucleotide is hybridized to a first nucleic acid molecule covalently attached to a specific binding agent (e.g., a protein-specific binding agent). In embodiments, the specific binding agent is bound to a protein target. In embodiments, the first circular polynucleotide includes a retarding agent. In embodiments, the second circular polynucleotide is hybridized to a second nucleic acid molecule. In embodiments, the second nucleic acid molecule is an endogenous nucleic acid molecule.

In embodiments, the first nucleic acid molecule includes an RNA nucleic acid sequence. In embodiments, the first nucleic acid molecule includes a DNA nucleic acid sequence. In embodiments, the first nucleic acid molecule is an RNA molecule. In embodiments, the first nucleic acid molecule is a DNA molecule. In embodiments, the second nucleic acid molecule includes an RNA nucleic acid sequence. In embodiments, the second nucleic acid molecule includes a DNA nucleic acid sequence. In embodiments, the second nucleic acid molecule is an RNA molecule. In embodiments, the second nucleic acid molecule is a DNA molecule.

In embodiments, the first nucleic acid molecule is about 2 to about 500 nucleotides. In embodiments, the first nucleic acid molecule is about 5 to 50 nucleotides. In embodiments, the first nucleic acid molecule is about 50 to about 120 nucleotides. In embodiments, the first nucleic acid molecule is about 100 to about 300 nucleotides. In embodiments, the first nucleic acid molecule is about 50 to about 500 nucleotides. In embodiments, the first nucleic acid molecule is about 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 nucleotides. In embodiments, the first nucleic acid molecule is about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110 nucleotides. In embodiments, the first nucleic acid molecule is about 1 to 3 kb, and only a portion of that target (e.g., 50 to 100 nucleotides) is sequenced. In embodiments, the first nucleic acid molecule is about 1 to 2 kb. In embodiments, the first nucleic acid molecule is less than 1 kb. In embodiments, the first nucleic acid molecule is about 500 nucleotides, about 200 nucleotides, or about 100 nucleotides. In embodiments, the first nucleic acid molecule is less than 100 nucleotides.

In embodiments, the second nucleic acid molecule is about 2 to about 500 nucleotides. In embodiments, the second nucleic acid molecule is about 5 to 50 nucleotides. In embodiments, the second nucleic acid molecule is about 50 to about 120 nucleotides. In embodiments, the second nucleic acid molecule is about 100 to about 300 nucleotides. In embodiments, the second nucleic acid molecule is about 50 to about 500 nucleotides. In embodiments, the second nucleic acid molecule is about 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 nucleotides. In embodiments, the second nucleic acid molecule is about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110 nucleotides. In embodiments, the second nucleic acid molecule is about 1 to 3 kb, and only a portion of that target (e.g., 50 to 100 nucleotides) is sequenced. In embodiments, the second nucleic acid molecule is about 1 to 2 kb. In embodiments, the second nucleic acid molecule is less than 1 kb. In embodiments, the second nucleic acid molecule is about 500 nucleotides, about 200 nucleotides, or about 100 nucleotides. In embodiments, the second nucleic acid molecule is less than 100 nucleotides.

In an aspect provided herein are kits for use in accordance with any of the compounds, compositions, or methods disclosed herein, and including one or more elements thereof. In embodiments, a kit includes labeled nucleotides including differently labeled nucleotides, enzymes, buffers, oligonucleotides, and related solvents and solutions. In embodiments, the kit includes one or more oligonucleotides (e.g., an oligonucleotide as described herein). The kit may also include a template nucleic acid (DNA and/or RNA), one or more primer polynucleotides, nucleoside triphosphates (including, e.g., deoxyribonucleotides, dideoxynucleotides, ribonucleotides, labeled nucleotides, and/or modified nucleotides), buffers, salts, and/or labels (e.g., fluorophores). In embodiments, the kit includes components useful for circularizing template polynucleotides using a ligation enzyme (e.g., CircLigase™ enzyme, Taq DNA Ligase, HiFi Taq DNA Ligase, T4 ligase, SplintR® ligase, or Ampligase™ DNA Ligase). For example, such a kit further includes the following components: (a) reaction buffer for controlling pH and providing an optimized salt composition for a ligation enzyme (e.g., CircLigase™ enzyme, Taq DNA Ligase, HiFi Taq DNA Ligase, T4 ligase, SplintR® ligase, or Ampligase DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label. In embodiments, the kit includes a modified terminal deoxynucleotidyl transferase (TdT) enzyme.

In embodiments, the kit includes a single-stranded polynucleotide described herein. In embodiments, the single-stranded polynucleotide is capable of hybridizing to a first nucleic acid molecule described herein. In embodiments, the single-stranded polynucleotide is capable of hybridizing to a first nucleic acid molecule covalently attached to a specific binding agent described herein. In embodiments, the single-stranded polynucleotide is capable of hybridizing to a second nucleic acid molecule described herein. In embodiments, the single-stranded polynucleotide described herein (i.e., the single-stranded polynucleotide capable of independently hybridizing to the first nucleic acid molecule or the second nucleic acid molecule) is a circularizable oligonucleotide.

In embodiments, the single-stranded polynucleotide includes an RNA nucleic acid sequence. In embodiments, the single-stranded polynucleotide includes a DNA nucleic acid sequence. In embodiments, the single-stranded polynucleotide is about 50 to about 500 nucleotides. In embodiments, the single-stranded polynucleotide is about 50 to about 300 nucleotides. In embodiments, the single-stranded polynucleotide is about 80 to about 300 nucleotides. In embodiments, the single-stranded polynucleotide is about 50 to about 150 nucleotides. In embodiments, the single-stranded polynucleotide is about or more than about 40, 50, 75, 100, 125, 150, 175, 200, 250, 300, 350, 400, or 500 nucleotides. In embodiments, the single-stranded polynucleotide is less than about 50, 75, 100, 125, 150, 175, 200, 250, 300, 350, 400, or 500 nucleotides. In embodiments, the single-stranded polynucleotide is about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 nucleotides. In embodiments, the single-stranded polynucleotide is about 100 nucleotides. In embodiments, the single-stranded polynucleotide is about 115 nucleotides. In embodiments, the single-stranded polynucleotide is about 120 nucleotides. In embodiments, the single-stranded polynucleotide is about 130 nucleotides. In embodiments, the single-stranded polynucleotide is about 135 nucleotides. In embodiments, the single-stranded polynucleotide is about 140 nucleotides. In embodiments, the single-stranded polynucleotide is about 145 nucleotides.

The single-stranded polynucleotide includes a first hybridization sequence and a second hybridization sequence. In embodiments, the first end of the single-stranded polynucleotide as described herein includes the first hybridization sequence. In embodiments, the second end of the single-stranded polynucleotide as described herein includes the second hybridization sequence. In embodiments, the first hybridization sequence includes about 5 to about 20 nucleotides. In embodiments, the first hybridization sequence includes about 15 to about 20 nucleotides. In embodiments, the first hybridization sequence includes about 20 to about 50 nucleotides. In embodiments, the second hybridization sequence includes about 5 to about 20 nucleotides. In embodiments, the second hybridization sequence includes about 15 to about 20 nucleotides. In embodiments, the second hybridization sequence includes about 20 to about 50 nucleotides. In embodiments, the first hybridization sequence and second hybridization sequence include about 35 to 40 nucleotides in length to maximize specificity. In embodiments, the first hybridization sequence and second hybridization sequence include about 5 to about 20 nucleotides. In embodiments, the first hybridization sequence and second hybridization sequence include about 15 to about 20 nucleotides. In embodiments, the first hybridization sequence and second hybridization sequence include about 20 to about 50 nucleotides. In embodiments, the first hybridization sequence and second hybridization sequence are about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In embodiments, the first hybridization sequence and second hybridization sequence include a single stranded polynucleotide that is at least 50% complementary, at least 75% complementary, at least 85% complementary, at least 90% complementary, at least 95% complementary, at least 98%, at least 99% complementary, or 100% complementary to a portion of a target polynucleotide. In embodiments, the first hybridization sequence is a flanking-target region. In embodiments, the second hybridization sequence is a flanking-target region. In embodiments, the length of the first hybridization sequence and second hybridization sequence are the same length (e.g., both the first and the second hybridization sequences are each about 15 nucleotides). In embodiments, the length of the first hybridization sequence and second hybridization sequence are different lengths (e.g., the first hybridization sequence is about 10 nucleotides and the second hybridization sequence is about 20 nucleotides). In embodiments, an asymmetric single-stranded polynucleotide (i.e., a single-stranded polynucleotide having a first hybridization sequence and second hybridization sequence that are different lengths) may be advantageous in preventing non-specific hybridization. In embodiments, the total length of the first hybridization sequence and second hybridization sequence combined is about 25, 30, 35, or 40 nucleotides. In embodiments, the combined length of the first hybridization sequence and the second hybridization sequence is about 20, 25, 30, 35, 40, 45, 50, 55, 60, 65, 70, 75, 80, 85, 90, 95, 100, 105, 110, 115, 120, 125, 130, 135, 140, 145, or 150 nucleotides.

In embodiments, the kit includes an oligonucleotide or a plurality of oligonucleotides. In embodiments, each oligonucleotide (e.g., each oligonucleotide of a plurality of oligonucleotides) includes a primer binding sequence (i.e., a sequence complementary to a primer, such as an amplification or sequencing primer). In embodiments, the first oligonucleotide and the second oligonucleotide each independently include a primer binding sequence. In embodiments, the first oligonucleotide and the second oligonucleotide each independently include a primer binding sequence, wherein the primer binding sequences are the same. In embodiments, the first oligonucleotide and the second oligonucleotide each independently include a primer binding sequence, wherein the primer binding sequences are different. In embodiments, the circularizable oligonucleotide includes a primer binding sequence. In embodiments, the oligonucleotide is a circularizable oligonucleotide.

In embodiments, each oligonucleotide includes about 50 to about 150 nucleotides. In embodiments, the first oligonucleotide and the second oligonucleotide each include about 50 to about 150 nucleotides. In embodiments, the circularizable oligonucleotide (e.g., a single-stranded polynucleotide described herein) includes about 50 to about 150 nucleotides. In embodiments, each oligonucleotide includes about 50 to about 300 nucleotides. In embodiments, the first oligonucleotide and the second oligonucleotide each include about 50 to about 300 nucleotides. In embodiments, the circularizable oligonucleotide (e.g., a single-stranded polynucleotide described herein) includes about 50 to about 300 nucleotides. In embodiments, the first oligonucleotide and the second oligonucleotide each include about 50 to about 300 nucleotides. In embodiments, each oligonucleotide includes about or more than about 50, 75, 100, 125, 150, 175, 200, 250, 300, 350, 400, or 500 nucleotides. In embodiments, the first oligonucleotide and the second oligonucleotide each include about or more than about 50, 75, 100, 125, 150, 175, 200, 250, 300, 350, 400, or 500 nucleotides. In embodiments, the circularizable oligonucleotide (e.g., a single-stranded polynucleotide described herein) includes about or more than about 50, 75, 100, 125, 150, 175, 200, 250, 300, 350, 400, or 500 nucleotides. In embodiments, each oligonucleotide includes less than about 50, 75, 100, 125, 150, 175, 200, 250, 300, 350, 400, or 500 nucleotides. In embodiments, the first oligonucleotide and the second oligonucleotide each include less than about 50, 75, 100, 125, 150, 175, 200, 250, 300, 350, 400, or 500 nucleotides. In embodiments, the circularizable oligonucleotide (e.g., a single-stranded polynucleotide described herein) includes less than about 50, 75, 100, 125, 150, 175, 200, 250, 300, 350, 400, or 500 nucleotides.

In embodiments, the circularizable oligonucleotide (e.g., a single-stranded polynucleotide described herein) includes a spacer sequence (e.g., an optional spacer sequence). In embodiments, the length of the spacer sequence is the combined length of the sequence complementary to the second hybridization sequence and the sequence complementary to the third hybridization sequence. In embodiments, the spacer sequence is about 5 to about 75 nucleotides in length. In embodiments, the spacer sequence is about 10 to about 150 nucleotides in length. In embodiments, the spacer sequence is about 5, 10, 15, 20, 25, 30, 40, 50, 60, 75, 100, 125, or 150 nucleotides in length.

In embodiments, the first hybridization sequence of the oligonucleotide is greater than 30 nucleotides. In embodiments, the first hybridization sequence of the oligonucleotide is about 5 to about 35 nucleotides in length. In embodiments, the first hybridization sequence of the oligonucleotide is about 5 to about 20 nucleotides in length. In embodiments, the first hybridization sequence is about 12 to 15 nucleotides in length. In embodiments, the first hybridization sequence is about 35 to 40 nucleotides in length to maximize specificity. In embodiments, the first hybridization sequence is greater than 12 nucleotides in length. In embodiments, the first hybridization sequence is about 5, about 10, about 15, about 20, about 25, about 30, or about 35 nucleotides in length.

In embodiments, the second hybridization sequence of the oligonucleotide is greater than 30 nucleotides. In embodiments, the second hybridization sequence of the oligonucleotide is about 5 to about 35 nucleotides in length. In embodiments, the second hybridization sequence of the oligonucleotide is about 5 to about 20 nucleotides in length. In embodiments, the second hybridization sequence is about 12 to 15 nucleotides in length. In embodiments, the second hybridization sequence is about 35 to 40 nucleotides in length to maximize specificity. In embodiments, the second hybridization sequence is greater than 12 nucleotides in length. In embodiments, the second hybridization sequence is about 5, about 10, about 15, about 20, about 25, about 30, or about 35 nucleotides in length.

In embodiments, the length of the second hybridization sequence is less than the length of the first hybridization sequence. In embodiments, the length of the second hybridization sequence is about 5% to 50% the length of the first hybridization sequence. In embodiments, the length of the second hybridization sequence is about 5% to 25% the length of the first hybridization sequence. In embodiments, the length of the second hybridization sequence is about 30% to 50% the length of the first hybridization sequence. In embodiments, the length of the second hybridization sequence is about 40% to 50% the length of the first hybridization sequence. In embodiments, the length of the second hybridization sequence is about 5%, about 10%, about 15%, about 20%, about 25%, about 30%, about 35%, about 40%, about 45%, or about 50% the length of the first hybridization sequence.

In embodiments, the combined length of the first hybridization sequence and the second hybridization sequence is about 20 to about 150 nucleotides. In embodiments, the combined length of the first hybridization sequence and the second hybridization sequence is about 50 to about 100 nucleotides. In embodiments, the combined length of the first hybridization sequence and the second hybridization sequence is about 20, about 30, about 40, about 50, about 75, about 100, about 125, or about 150 nucleotides.

In embodiments, each oligonucleotide includes about 50 to about 150 nucleotides. In embodiments, each oligonucleotide includes about 50 to about 300 nucleotides. In embodiments, each oligonucleotide includes about 50 to about 500 nucleotides. In embodiments, each oligonucleotide includes about or more than about 50, 75, 100, 125, 150, 175, 200, 250, 300, 350, 400, or 500 nucleotides. In embodiments, each oligonucleotide includes less than about 50, 75, 100, 125, 150, 175, 200, 250, 300, 350, 400, or 500 nucleotides.

In embodiments, each oligonucleotide (e.g., each oligonucleotide of a plurality of oligonucleotides targeting a sequence of a target polynucleotide (e.g., the first nucleic acid molecule described herein or second nucleic acid molecule described herein) includes a barcode sequence. In embodiments, the first oligonucleotide includes a barcode sequence. In embodiments, the second oligonucleotide includes a barcode sequence. In embodiments, the circularizable oligonucleotide includes a barcode sequence.

In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. Attachment of the barcode sequence (via binding of a specific binding agent described herein conjugated to the barcode sequence) to a protein or nucleic acid of interest (i.e., the target) may associate the barcode sequence with the protein or nucleic acid of interest. The barcode may then be used to identify the protein or nucleic acid of interest during sequencing, even when other proteins or nucleic acids of interest (e.g., including different oligonucleotide barcodes) are present. In embodiments, the barcode consists only of a unique barcode sequence. In embodiments, the 5′ end of a barcoded oligonucleotide is phosphorylated. In embodiments, the barcode is known (i.e., the nucleic sequence is known before sequencing) and is sorted into a basis-set according to their Hamming distance. Oligonucleotide barcodes (e.g., barcode sequences included in an oligonucleotide) can be associated with a target of interest by knowing, a priori, the target of interest, such as a gene or protein. In embodiments, the barcodes further include one or more sequences capable of specifically binding a gene or nucleic acid sequence of interest. For example, in embodiments, the barcode includes a sequence capable of hybridizing to mRNA, e.g., one containing a poly-T sequence (e.g., having several T's in a row, e.g., 4, 5, 6, 7, 8, or more T's). In embodiments, the first hybridization sequence (e.g., the first end of the single-stranded polynucleotide described herein) and/or the second hybridization sequence (e.g., the second end of the single-stranded polynucleotide described herein) may be used as a barcode sequence (e.g., sequencing all or a portion of the second hybridization sequence) to identify the circular oligonucleotide.

In embodiments, the barcode is included as part of an oligonucleotide of longer sequence length, such as a primer or a random sequence (e.g., a random N-mer). In embodiments, the barcode contains random sequences to increase the mass or size of the oligonucleotide tag. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides. In embodiments, each barcode sequence is selected from a known set of barcode sequences. In embodiments, each of the known set of barcode sequences is associated with a target hybridization sequence from a known set of target hybridization sequences. In embodiments, a first barcode sequence is associated with a first target hybridization sequence, and wherein a second barcode sequence is associated with a second target hybridization sequence (e.g., wherein the second target hybridization sequence is included in an oligonucleotide targeting a different target nucleic acid than the first target hybridization sequence). In embodiments, the same barcode sequence is associated with a plurality of oligonucleotides targeting different sequences of the same target nucleic acid (e.g., the same target polynucleotide).

In embodiments, the barcode sequence is selected from a known set of barcode sequences. In embodiments, each barcode sequence is unique within the known set of barcodes. In embodiments, the barcodes are selected to form a known set of barcodes, e.g., the set of barcodes may be distinguished by a particular Hamming distance.

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, the polynucleotide (e.g., the single-stranded polynucleotide described herein) includes a barcode nucleotide. A barcode nucleotide refers to a single nucleotide which may serve as a differentiating feature among targets. Detecting four different targets using a single nucleotide as a barcode may involve the use of a common primer and the incorporation of differently colored labeled nucleotides into the primer, rendering simultaneous detection of multiple targets. For example, one may bind a common primer to each of the four separate targets (e.g., amplification products arising from four separate target molecules). This common primer is designed to hybridize to a specific region shared among the targets, serving as a starting point for the subsequent incorporation of nucleotides. With a polymerase, differently colored labeled nucleotides are incorporated into the newly synthesized DNA strand opposite the barcode nucleotide. Each of the four types of nucleotides (adenine, thymine, cytosine, and guanine) is tagged with a unique fluorescent dye, with each dye emitting a distinct color upon excitation. For instance, adenine might be tagged with a green dye, thymine with blue, cytosine with red, and guanine with yellow. As the primer is extended, a colored nucleotide is incorporated to a position complementary to the barcode nucleotide. Detection is then based on the color emitted upon fluorescence excitation. For example, if the barcode nucleotide is adenine, then the complementary thymine, labeled with a blue fluorophore, is incorporated into the extending strand. The presence of the target adenine is then identified by the emission of a blue fluorescence signal. This color-coded system allows for the distinct identification of each of the four targets based on the specific fluorescence emitted by the incorporated nucleotides.

In embodiments, the first nucleic acid molecule includes a target nucleic acid (i.e., the target polynucleotide). In embodiments, the second nucleic acid molecule includes a target nucleic acid (i.e., the target polynucleotide). In embodiments, the target nucleic acid (i.e., the target polynucleotide) includes a nucleic acid sequence encoding a TCR alpha chain, a TCR beta chain, a TCR delta chain, a TCR gamma chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

In embodiments, the target polynucleotide includes a cancer-associated gene nucleic acid sequence, a viral nucleic acid sequence, a bacterial nucleic acid sequence, or a fungal nucleic acid sequence. In embodiments, the cancer-associated gene is a nucleic acid sequence identified within The Cancer Genome Atlas Program, accessible at www.cancer.gov/tcga.

In embodiments, the target polynucleotide can include any polynucleotide of interest. The polynucleotide can include DNA, RNA, peptide nucleic acid, morpholino nucleic acid, locked nucleic acid, glycol nucleic acid, threose nucleic acid, mixtures thereof, and hybrids thereof. In embodiments, the polynucleotide is obtained from one or more source organisms. In some embodiments, the polynucleotide can include a selected sequence or a portion of a larger sequence. In embodiments, sequencing a portion of a polynucleotide or a fragment thereof can be used to identify the source of the polynucleotide. With reference to nucleic acids, polynucleotides and/or nucleotide sequences a “portion,” “fragment” or “region” can be at least 5 consecutive nucleotides, at least 10 consecutive nucleotides, at least 15 consecutive nucleotides, at least 20 consecutive nucleotides, at least 25 consecutive nucleotides, at least 50 consecutive nucleotides, at least 100 consecutive nucleotides, or at least 150 consecutive nucleotides.

In embodiments, the entire sequence of the target polynucleotide is about 1 to 3 kb, and only a portion of that target (e.g., 50 to 100 nucleotides) is sequenced. In embodiments, the target polynucleotide is about 1 to 3 kb. In embodiments, the target polynucleotide is about 1 to 2 kb. In embodiments, the target polynucleotide is about 1 kb. In embodiments, the target polynucleotide is about 2 kb. In embodiments, the target polynucleotide is less than 1 kb. In embodiments, the target polynucleotide is about 500 nucleotides. In embodiments, the target polynucleotide is about 200 nucleotides. In embodiments, the target polynucleotide is about 100 nucleotides. In embodiments, the target polynucleotide is less than 100 nucleotides. In embodiments, the target polynucleotide is about 5 to 50 nucleotides.

In embodiments, each oligonucleotide includes a blocking moiety at a 3′ end (e.g., at the 3′ end of each oligonucleotide of a plurality of oligonucleotides). In embodiments, the first oligonucleotide and/or the second oligonucleotide includes a blocking moiety at the 3′ end. In embodiments, the first oligonucleotide and the second oligonucleotide include a blocking moiety at the 3′ end. In embodiments, a terminal nucleotide of the first oligonucleotide includes a blocking moiety. In embodiments, a terminal nucleotide of the second oligonucleotide includes a blocking moiety. In embodiments, the blocking moiety is reversible. In embodiments, the blocking moiety is irreversible. In embodiments, the blocking moiety at the 3′ end (e.g., the 3′ blocking moiety) includes a reversible terminator. In embodiments, the 3′ blocking moiety includes a dideoxynucleotide triphosphate (e.g., a ddNTP).

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μDNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg2+, Mn2+, Zn2+, and Ca2+. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In embodiments, the buffered solution includes about 10 mM Tris, about 20 mM Tris, about 30 mM Tris, about 40 mM Tris, or about 50 mM Tris. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton X-100, about 0.025% Triton X-100, about 0.05% Triton X-100, about 0.1% Triton X-100, or about 0.5% Triton X-100. In embodiments, the buffered solution includes 20 mM Tris pH 8.0, 100 mM NaCl, 0.1 mM EDTA, 0.025% Triton X-100. In embodiments, the buffered solution includes 20 mM Tris pH 8.0, 150 mM NaCl, 0.1 mM EDTA, 0.025% Triton X-100. In embodiments, the buffered solution includes 20 mM Tris pH 8.0, 300 mM NaCl, 0.1 mM EDTA, 0.025% Triton X-100. In embodiments, the buffered solution includes 20 mM Tris pH 8.0, 400 mM NaCl, 0.1 mM EDTA, 0.025% Triton X-100. In embodiments, the buffered solution includes 20 mM Tris pH 8.0, 500 mM NaCl, 0.1 mM EDTA, 0.025% Triton X-100. In embodiments, the buffered solution includes about 0.002% Pluronic® F-127, about 0.01% Pluronic® F-127, about 0.02% Pluronic® F-127, about 0.05% Pluronic® F-127, about 0.1% Pluronic® F-127, about 0.2% Pluronic® F-127, about 0.3% Pluronic® F-127, about 0.4% Pluronic® F-127, about 0.5% Pluronic® F-127, about 0.6% Pluronic® F-127, about 0.7% Pluronic® F-127, about 0.8% Pluronic® F-127, about 0.9% Pluronic® F-127, about 1% Pluronic® F-127, about 1.1% Pluronic® F-127, about 1.2% Pluronic® F-127, about 1.3% Pluronic® F-127, about 1.4% Pluronic® F-127, about 1.5% Pluronic® F-127, about 1.6% Pluronic® F-127, about 1.7% Pluronic® F-127, about 1.8% Pluronic® F-127, about 1.9% Pluronic® F-127, or about 2% Pluronic® F-127.

In embodiments, the kit includes one or more sequencing reaction mixtures. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

In embodiments, the kit can further include one or more biological stain(s) (e.g., any of the biological stains as described herein). For example, the kit can further include eosin and hematoxylin. In other examples, the kit can include a biological stain such as acridine orange, Bismarck brown, carmine, coomassie blue, crystal violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In embodiments, the kit is designed for staining tissue samples for imaging and detecting target molecules (e.g., proteins) can be significantly expanded beyond the inclusion of fluorophores. For instance, the kit can include eosin and hematoxylin, which are classic histological stains. Eosin, a red dye, typically stains acidic components of the cell such as cytoplasmic proteins, while hematoxylin, a basic dye, binds to nucleic acids, coloring the cell nucleus blue. This combination is widely used in histopathology for detailed tissue structure visualization. Moreover, the kit can encompass stains such as acridine orange, a nucleic acid-selective fluorescent cationic dye, and Bismarck brown, which is often used for staining backgrounds in histological tissue sections. Carmine, another potential inclusion, is a natural red dye used for staining glycogen, while Coomassie blue is a popular choice for protein staining in gel electrophoresis. Crystal violet, a triarylmethane dye, can be included for staining cell walls and nuclei, and DAPI, a fluorescent stain that binds strongly to A-T rich regions in DNA, is useful in fluorescence microscopy. Ethidium bromide, a fluorescent intercalator, is also a valuable addition for its role in nucleic acid staining, especially in gel electrophoresis. Further, the kit can include acid fuchsine, used in Masson's trichrome stain; Hoechst stains, which are cell-permeable, DNA-specific blue fluorescent dyes; and iodine, commonly used in Gram staining and for staining starch in plant cells. Methyl green and methylene blue, both traditional histological stains, can be included for their affinity towards nucleic acids. Neutral red, a vital stain that accumulates in lysosomes, Nile blue and Nile red, both used for staining lipids, and osmium tetroxide, a heavy metal stain for lipid bilayers in electron microscopy, can be part of the kit. Propidium iodide, a popular red-fluorescent nuclear and chromosome counterstain, along with rhodamine, may be utilized. Safranin, commonly used in Gram staining, can be included for its ability to stain cell components like nuclei, cytoplasm, and cell walls in various colors, enhancing the contrast and detail in tissue imaging.

To facilitate visualization, biological samples can be stained using a wide variety of stains and staining techniques. In embodiments, for example, the kit may include any number of stains, including but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetraoxide, propidium iodide, rhodamine, safranine and/or an immunofluorescence stain.

### III. Methods

In an aspect is provided a method of profiling a sample (e.g., a cell). In embodiments, the method includes determining information (e.g., gene and protein expression) about the transcriptome of an organism thus elucidating subcellular substances and processes while gaining valuable spatial localization information within a cell. In embodiments, the method includes simultaneously sequencing a plurality of nucleic acids, such as RNA transcripts, in situ within an optically resolved volume of a sample (e.g., a voxel). RNA transcripts are responsible for the process of converting DNA into an organism's phenotype, thus by determining the types and quantity of RNA present in a sample (e.g., a cell), it is possible to assign a phenotype to the cell. RNA transcripts include coding RNA and non-coding RNA molecules, such as messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target is pre-mRNA. In embodiments, the target is heterogeneous nuclear RNA (hnRNA). In embodiments, the targets are proteins or carbohydrates. In embodiments, the targets are proteins. In embodiments, the targets are carbohydrates. In embodiments when the target are proteins and/or carbohydrates, the method includes contacting the proteins with a specific binding reagent or specific binding agent, wherein the specific binding reagent or specific binding agent includes an oligonucleotide barcode. In embodiments, the specific binding reagent or specific binding agent includes a protein-specific binding agent. In embodiments, the protein-specific binding agent includes an antibody, single-chain Fv fragment (scFv), antibody fragment-antigen binding (Fab), or an aptamer. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. In embodiments, the specific binding reagent interacts (e.g., contacts, or binds) with one or more specific binding reagents on the cell surface. Carbohydrate-specific antibodies are known in the art, see for example Kappler, K., Hennet, T. Genes Immun 21, 224-239 (2020).

In an aspect is provided a method for differentially amplifying polynucleotides in or on a cell or tissue. In embodiments, the method includes (i) contacting a cell or tissue with a plurality of polymerases and deoxynucleotide triphosphates (dNTPs) and (ii) amplifying a first circular polynucleotide including a first sequence to generate an amplification product including a first number of copies of the first sequence and amplifying a second circular polynucleotide including a second sequence to generate an amplification product including a second number of copies of the second sequence; wherein the first number is detectably less than the second number, wherein the first circular polynucleotide is hybridized to a first nucleic acid molecule covalently attached to a specific binding agent, wherein the first circular polynucleotide includes a retarding agent; and the second circular polynucleotide is hybridized to a second nucleic acid molecule.

In embodiments, the retarding agent is a modified nucleotide. In embodiments, the modified nucleotide is a 2′-O-methyl ribonucleic acid (2′-OMeRNA) nucleotide, biotin-nucleotide, 2′-fluoro ribonucleic acid (2′-F RNA) nucleotide, locked nucleic acid (LNA) nucleotide, or phosphorothioate (PS) nucleotide. In embodiments, the modified nucleotide is a 2′OMe modified cytosine. In embodiments, the modified nucleotide is a 2′OMe modified adenine. In embodiments, the modified nucleotide is a 2′OMe modified guanine. In embodiments, the modified nucleotide is a 2′OMe modified uracil. In embodiments, the modified nucleotide is a 2′OMe modified thymine.

In embodiments, the circular polynucleotide includes one or more modified nucleotides. In embodiments, the modified nucleotide includes a modification to the sugar. In embodiments, the nucleotide includes a modification to the base. In embodiments, the circular polynucleotide includes a modified nucleotide adjacent to the primer binding sequence (e.g., immediately 5′ to the primer binding sequence). In embodiments, the modified nucleotide is a biotin nucleotide (e.g., a nucleotide including a biotin moiety). For example, the modified nucleotide includes a biotin moiety attached to the nucleobase of the nucleotide. In embodiments, the modified nucleotide has the structure:

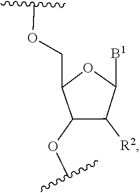

wherein B¹ is a nucleobase, R² is halogen (e.g., F or Cl), —O—[C₁-C₄ alkyl]-O—[C₁-C₄ alkyl](e.g., —CH₂—CH₂—O—CH₃), or —O—C₁-C₄ alkyl (e.g., —O—CH₃), and “ ” represents the attachment points to the remainder of the circular polynucleotide. In embodiments, R² is F. In embodiments, the modified nucleotide has the structure:

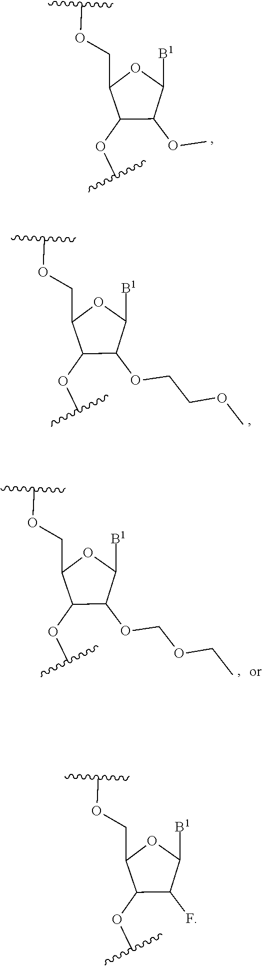

In embodiments, B¹ is

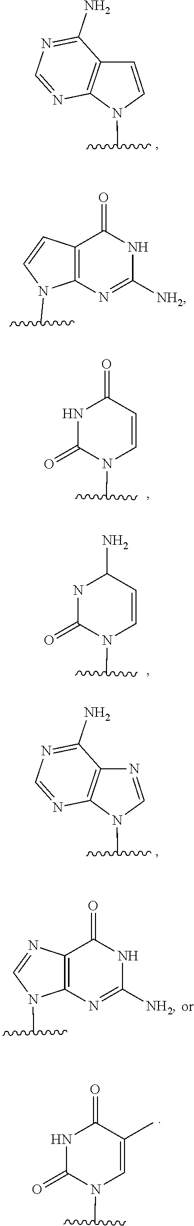

In embodiments, B¹ is

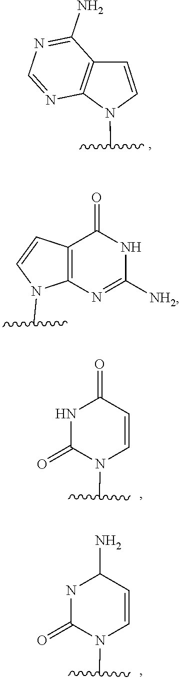

In embodiments, B¹ is

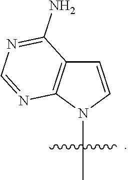 [SMILES: *n1ccc2c(N)ncnc21]

In embodiments, B¹ is

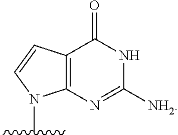 [SMILES: *n1ccc2c(=O)[nH]c(N)nc21]

In embodiments, B¹ is

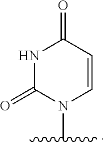 [SMILES: *n1ccc(=O)[nH]c1=O]

In embodiments, B¹ is

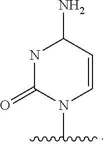 [SMILES: *N1C=CC(N)NC1=O]

In embodiments, B¹ is

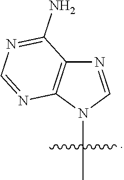 [SMILES: *n1cnc2c(N)ncnc21]

In embodiments, B¹ is

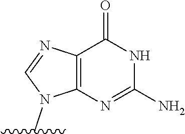 [SMILES: *n1cnc2c(=O)[nH]c(N)nc21]

embodiments, B¹ is

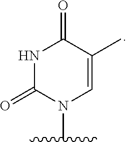 [SMILES: *n1cc(C)c(=O)[nH]c1=O]

In embodiments, B¹ is

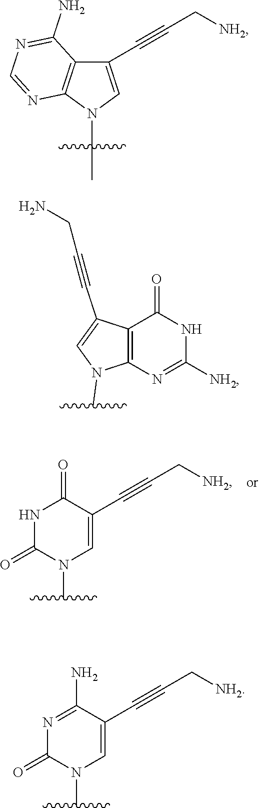 [SMILES: *n1cc(C#CCN)c2c(N)ncnc21 ; *n1cc(C#CCN)c2c(=O)[nH]c(N)nc21 ; *n1cc(C#CCN)c(=O)[nH]c1=O ; *n1cc(C#CCN)c(N)nc1=O]

In embodiments, B¹ is

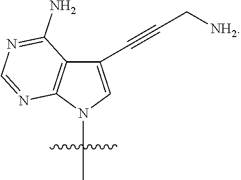 [SMILES: *n1cc(C#CCN)c2c(N)ncnc21]

In embodiments, B¹ is

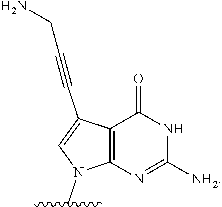 [SMILES: *n1cc(C#CCN)c2c(=O)[nH]c(N)nc21]

In embodiments, B¹ is

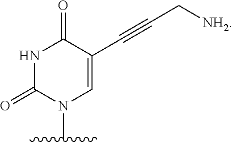 [SMILES: *n1cc(C#CCN)c(=O)[nH]c1=O]

In embodiments, B¹ is

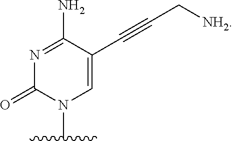 [SMILES: *n1cc(C#CCN)c(N)nc1=O]

In embodiments, B¹ is

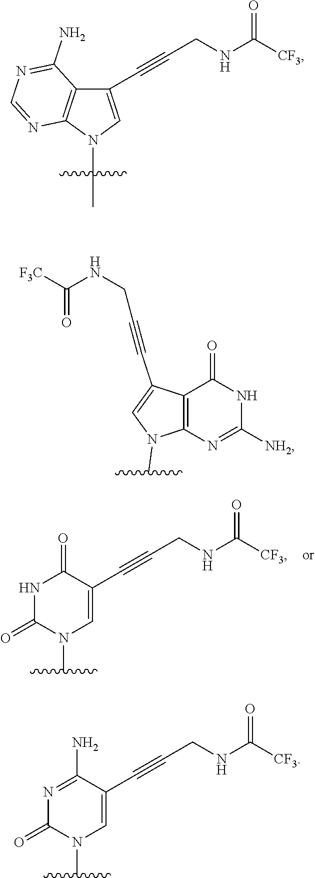 [SMILES: *n1cc(C#CCNC(=O)C(F)(F)F)c2c(N)ncnc21 ; *n1cc(C#CCNC(=O)C(F)(F)F)c2c(=O)[nH]c(N)nc21 ; *n1cc(C#CCNC(=O)C(F)(F)F)c(=O)[nH]c1=O ; *n1cc(C#CCNC(=O)C(F)(F)F)c(N)nc1=O]

In embodiments, B¹ is

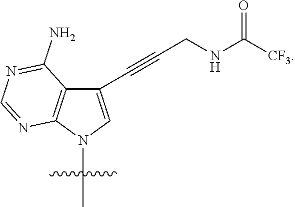 [SMILES: *n1cc(C#CCNC(=O)C(F)(F)F)c2c(N)ncnc21]

In embodiments, B¹ is

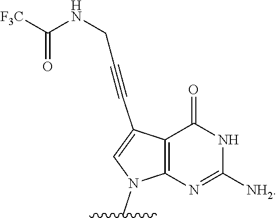 [SMILES: *n1cc(C#CCNC(=O)C(F)(F)F)c2c(=O)[nH]c(N)nc21]

In embodiments, B¹ is

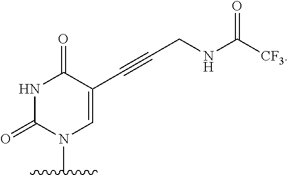 [SMILES: *n1cc(C#CCNC(=O)C(F)(F)F)c(=O)[nH]c1=O]

In embodiments, B¹ is

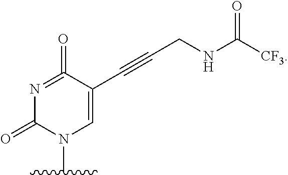

In embodiments, B¹ is

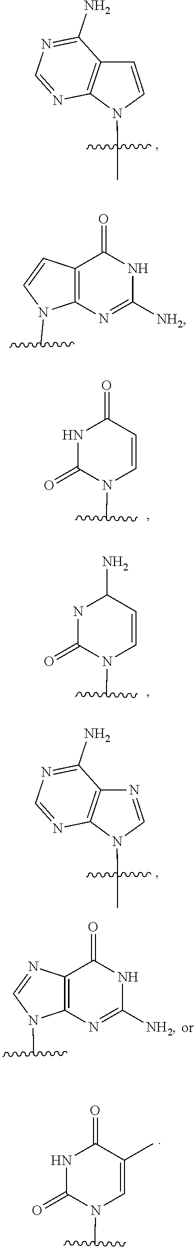

In embodiments, B¹ is

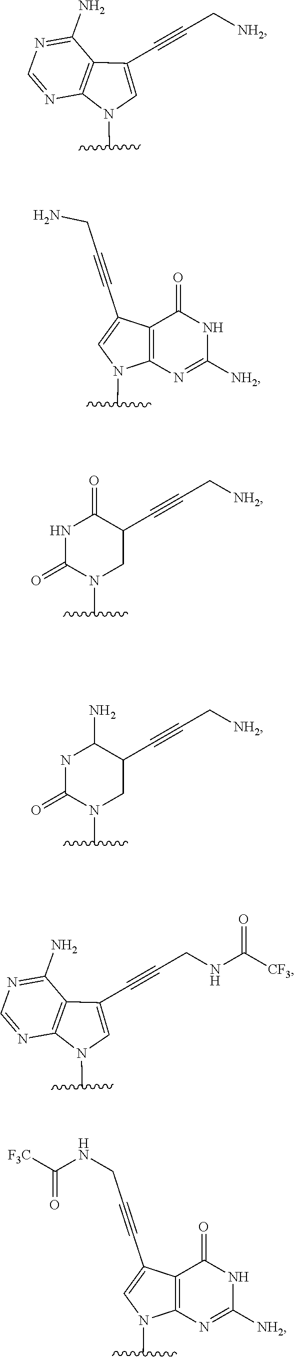 [SMILES: *n1cc(C#CCN)c2c(N)ncnc21 ; *n1cc(C#CCN)c2c(=O)[nH]c(N)nc21 ; *N1CC(C#CCN)C(=O)NC1=O ; *N1CC(C#CCN)C(N)NC1=O ; *n1cc(C#CCNC(=O)C(F)(F)F)c2c(N)ncnc21 ; *n1cc(C#CCNC(=O)C(F)(F)F)c2c(=O)[nH]c(N)nc21]

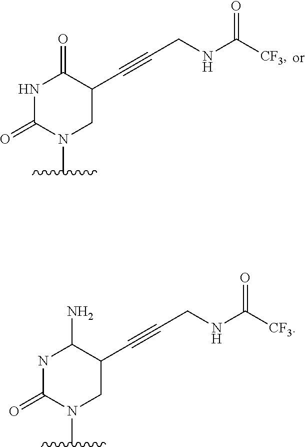 [SMILES: *N1CC(C#CCNC(=O)C(F)(F)F)C(=O)NC1=O ; *N1CC(C#CCNC(=O)C(F)(F)F)C(N)NC1=O]

In embodiments, B¹ is —B-L¹⁰⁰-R⁴. B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof. L¹⁰⁰ is a covalent linker; and R⁴ is a retarding moiety.

In embodiments, B is

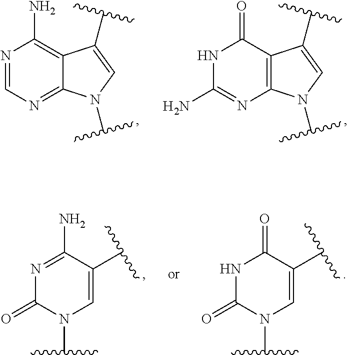 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

In embodiments, B is

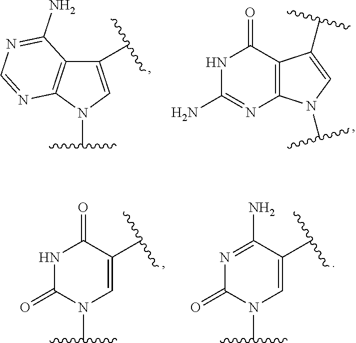 [SMILES: *c1cn(*)c2ncnc(N)c12 ; *c1cn(*)c2nc(N)[nH]c(=O)c12 ; *c1cn(*)c(=O)[nH]c1=O ; *c1cn(*)c(=O)nc1N]

In embodiments, B is

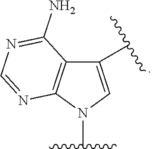 [SMILES: *c1cn(*)c2ncnc(N)c12]

In embodiments, B is

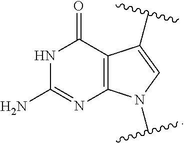 [SMILES: *c1cn(*)c2nc(N)[nH]c(=O)c12]

In embodiments, B is

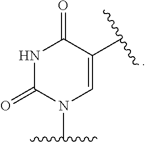 [SMILES: *c1cn(*)c(=O)[nH]c1=O]

In embodiments, B is

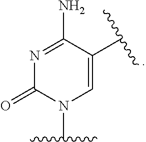 [SMILES: *c1cn(*)c(=O)nc1N]

In embodiments, B is

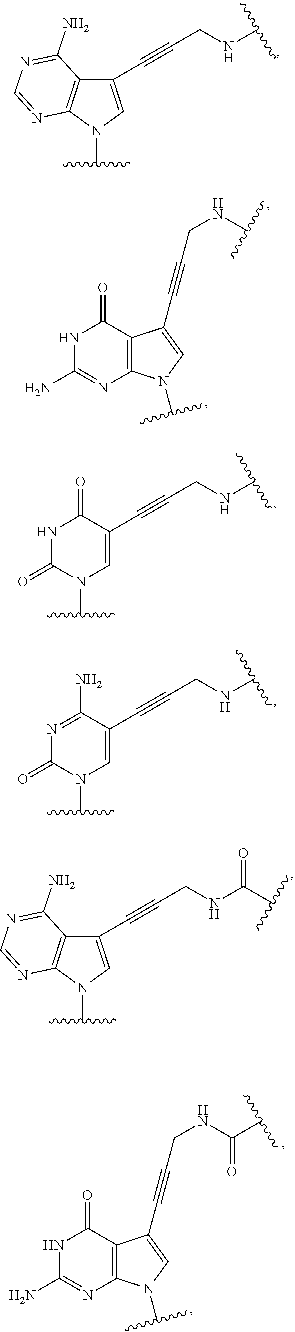 [SMILES: *NCC#Cc1cn(*)c2ncnc(N)c12 ; *NCC#Cc1cn(*)c2nc(N)[nH]c(=O)c12 ; *NCC#Cc1cn(*)c(=O)[nH]c1=O ; *NCC#Cc1cn(*)c(=O)nc1N ; *C(=O)NCC#Cc1cn(*)c2ncnc(N)c12 ; *C(=O)NCC#Cc1cn(*)c2nc(N)[nH]c(=O)c12]

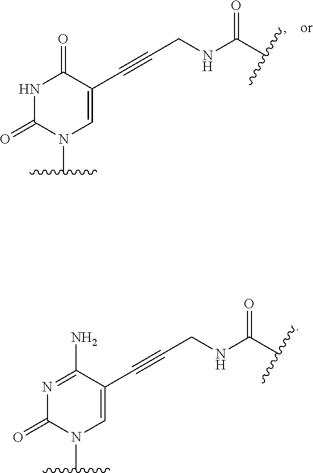 [SMILES: *C(=O)NCC#Cc1cn(*)c(=O)[nH]c1=O ; *C(=O)NCC#Cc1cn(*)c(=O)nc1N]

In embodiments, B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof. In embodiments, B is a divalent cytosine or a derivative thereof. In embodiments, B is a divalent guanine or a derivative thereof. In embodiments, B is a divalent adenine or a derivative thereof. In embodiments, B is a divalent thymine or a derivative thereof. In embodiments, B is a divalent uracil or a derivative thereof. In embodiments, B is a divalent hypoxanthine or a derivative thereof. In embodiments, B is a divalent xanthine or a derivative thereof. In embodiments, B is a divalent 7-methylguanine or a derivative thereof. In embodiments, B is a divalent 5,6-dihydrouracil or a derivative thereof. In embodiments, B is a divalent 5-methylcytosine or a derivative thereof. In embodiments, B is a divalent 5-hydroxymethylcytosine or a derivative thereof. In embodiments, B is a divalent cytosine. In embodiments, B is a divalent guanine. In embodiments, B is a divalent adenine. In embodiments, B is a divalent thymine. In embodiments, B is a divalent uracil. In embodiments, B is a divalent hypoxanthine. In embodiments, B is a divalent xanthine. In embodiments, B is a divalent 7-methylguanine. In embodiments, B is a divalent 5,6-dihydrouracil. In embodiments, B is a divalent 5-methylcytosine. In embodiments, B is a divalent 5-hydroxymethylcytosine.

In embodiments, the modified nucleotide has the structure:

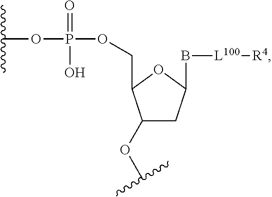

wherein B is a nucleobase, L¹⁰⁰ is a covalent linker, R⁴ is a retarding moiety, and “ [symbol] ” represents the attachment points to the remainder of the circular polynucleotide. In embodiments, R⁴ is a bioconjugate reactive moiety. In embodiments, R⁴ is an azide moiety. In embodiments, R⁴ is a biotin moiety. In embodiments, the modified nucleotide has the structure

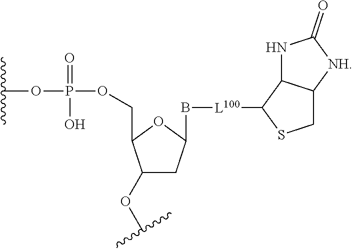

In embodiments, the modified nucleotide has the structure:

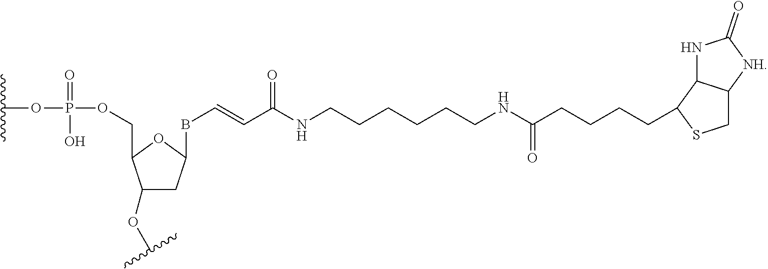

In embodiments, the modified nucleotide is 5-chloro-2′-deoxyuridine triphosphate, 7-deaza-2′-deoxyadenosine triphosphate, 5-fluoro-2′-deoxycytidine triphosphate, 7-deaza-2′-deoxyguanosine triphosphate, 7-Deaza-7-nitro-dATP, 7-deaza-7-nitro-dGTP, 5-hydroxy-dCTP, 5-hydroxy-dUTP, 5-ethynyl-deoxyuridine triphosphate, or 5′-(α-P-borano)deoxynucleosidetriphosphate.

In embodiments, the modified nucleotide includes a bioconjugate reactive moiety. In embodiments, the bioconjugate reactive moiety includes an amine moiety, aldehyde moiety, alkyne moiety, azide moiety, carboxylic acid moiety, dibenzocyclooctyne (DBCO) moiety, norbornene moiety, tetrazine moiety, epoxy moiety, isocyanate moiety, furan moiety, maleimide moiety, thiol moiety, or transcyclooctene (TCO) moiety. For example, the modified nucleotide may include a biotin moiety, wherein a streptavidin protein may be optionally introduced to sterically hinder amplification. In embodiments, the modified nucleotide includes an azide moiety. A blocking agent including a second bioconjugate reactive moiety (e.g., an alkynyl moiety or DBCO moiety) reacts with the bioconjugate reactive moiety and forms a bioconjugate linker that covalently links the modified nucleotide and blocking compound together.

In embodiments, the retarding agent includes one or more sequences which are recognized and bound by one or more retarding oligonucleotides. For example, during amplification (e.g., after 1-5 minutes of RCA) a plurality of oligonucleotides are introduced and hybridize to the amplification products. In embodiments, the retarding agent is an aptamer. In embodiments, the retarding agent is a blocking oligonucleotide bound to the circular polynucleotide (e.g., the first circular nucleotide). In embodiments, the oligonucleotides include a melting temperature (Tm), typically in the range of 60° C. to 70° C. The relatively higher Tm ensures that the oligonucleotides bind robustly to their target sequences and are not easily displaced by any strand displacement activity of the amplification enzyme. To achieve a Tm of 60° C. to 70° C., the oligonucleotides includes about 15-20 nucleotides in length, with a higher GC content to increase the stability of the duplex. In embodiments, the oligonucleotides include PNAs. Peptide Nucleic Acid (PNA) is a synthetic analog of DNA in which the sugar-phosphate backbone is replaced by a peptide-like backbone composed of repeating N-(2-aminoethyl)-glycine units. Despite this significant structural difference, PNA retains the ability to form complementary base pairs with DNA and RNA through Watson-Crick base pairing. The neutral peptide-like backbone of PNA does not have the negatively charged phosphate groups found in DNA and RNA. This lack of charge repulsion between PNA and its complementary nucleic acid strand results in stronger binding and higher affinity. The absence of electrostatic repulsion and the flexibility of the PNA backbone allow for more optimal base stacking interactions, contributing to the stability and higher melting temperature (Tm) of PNA-DNA or PNA-RNA duplexes. The “melting temperature” or “Tm” of a nucleic acid is defined as the temperature at which half of the helical structure of the nucleic acid is lost due to heating or other dissociation of the hydrogen bonding between base pairs, for example, by acid or alkali treatment, or the like. The Tm of a nucleic acid molecule depends on its length and on its base composition. Nucleic acid molecules rich in GC base pairs have a higher Tm than those having an abundance of AT base pairs. Separated complementary strands of nucleic acid spontaneously reassociate or anneal to form duplex nucleic acid when the temperature is lowered below the Tm. The highest rate of nucleic acid hybridization typically occurs approximately 25 degrees C. below the Tm. The Tm may be estimated using the following relationship: Tm=69.3+0.41(GC) % (Marmur et al. (1962) J. Mol. Biol. 5:109-118).

In embodiments, the retarding agent is a hairpin oligonucleotide. For example, the circular oligonucleotide may include a double-stranded region. In embodiments, the retarding agent is a hairpin oligonucleotide designed with a high melting temperature (Tm) in the range of 60° C. to 70° C. In embodiments, the hairpin oligonucleotide has a high GC content, typically between 50% and 70%, which enhances the thermal stability and binding strength of the double-stranded region. In embodiments, the hairpin oligonucleotide includes a stem of 8-12 base pairs and a loop of 4-8 nucleotides, creating a stable secondary structure that maintains its integrity and effectively slows amplification in the presence of strand-displacing enzymes. In embodiments, the hairpin oligonucleotide includes a stem including about 3, 4, 5, 6, 7, 8, 9, 10, 11, 12, 13, 14, 15, 16, 17, 18, 19, 20, 21, 22, 23, 24, 25, 26, 27, 28, 29, or 30 base pairs. In embodiments, the hairpin oligonucleotide includes a loop including about 4, 5, 6, 7, 8, 9, or 10 nucleotides. In embodiments, the hairpin oligonucleotide is designed to form a robust secondary structure with high stability, allowing it to resist displacement by phi29 DNA polymerase, thus providing a controlled modulation of the amplification process.

In embodiments, the circular oligonucleotide (e.g., a circular polynucleotide described herein) is about 100 to about 1000 nucleotides in length. In embodiments, the circular oligonucleotide is about 100, about 200, about 300, about 400, about 500, about 600, about 700, about 800, about 900, or about 1000 nucleotides in length. In embodiments, the circular oligonucleotide is greater than 1000 nucleotides in length. In embodiments, the circular oligonucleotide is about or more than about 100, 150, 200, 250, 300, 350, 400, 500, 750, 1000, or more nucleotides in length. In embodiments, the circular oligonucleotide includes a plurality of primer binding sequences. In embodiments, the circular oligonucleotide includes a plurality of different primer binding sequences.

In embodiments, the first polynucleotide is hybridized to a first nucleic acid molecule covalently attached to a protein-specific binding agent (e.g., an antibody-oligonucleotide, Ab-O, conjugate). In embodiments, the specific binding agent is a protein-specific binding agent. In embodiments, the protein-specific binding agent is an antibody, single domain antibody, single-chain Fv fragment (scFv), antibody fragment-antigen binding (Fab), affimer, or an aptamer.

In embodiments, the specific binding agent is an antibody, single-chain Fv fragment (scFv), antibody fragment-antigen binding (Fab), affimer, or an aptamer. In embodiments, the specific binding reagent is an antibody. In embodiments, the specific binding reagent is a single-chain Fv fragment (scFv). In embodiments, the specific binding reagent is an antibody fragment-antigen binding (Fab). In embodiments, the specific binding reagent is an affimer. In embodiments, the specific binding reagent is an aptamer.

In embodiments, the specific binding agent is an enzyme, enzyme mutant, peptide, Molecular Imprinted Polymer (MIP), DARPin (Designed Ankyrin Repeat Protein), peptoid, lectin, siRNA, or miRNA molecule. In embodiments, the specific binding agent is an enzyme. In embodiments, the specific binding agent is an enzyme mutant. In embodiments, the specific binding agent is a peptide. In embodiments, the specific binding agent is a Molecular Imprinted Polymer (MIP). In embodiments, the specific binding agent is a DARPin (Designed Ankyrin Repeat Protein). In embodiments, the specific binding agent is a peptoid. In embodiments, the specific binding agent is a lectin. In embodiments, the specific binding agent is an siRNA molecule. In embodiments, the specific binding agent is an miRNA molecule.

In embodiments, the specific binding agent is capable of binding to a cluster of differentiation (CD) marker, integrin, selectin, cadherin, cytokine receptor, chemokine receptor, Toll-like receptor (TLR), ion channel, transmembrane protein, lipoprotein, glycoprotein, cell surface protein, transport protein, intracellular organelle, or transcription factor. In embodiments, the intracellular organelle includes actin, carbohydrate, centrosomes and centrioles, chloroplasts (in plant cells and some protists), cytoskeleton, endoplasmic reticulum, endosome, golgi apparatus, intermediate filaments, lysosome, microfilaments, microtubules, mitochondria, nuclear envelope, nuclear pores, nucleoid, nucleolus, nucleus, peroxisome, phosphatidylserine, plasma membrane, ribosomes, rough endoplasmic reticulum, smooth endoplasmic reticulum, transferrin receptor, transport vesicles, and/or vacuoles. In embodiments, the biomolecule specific binding agent is capable of binding to a biomolecule in the mitogen-activated protein kinase (MAPK) pathway, PI3K/AKT/mTOR pathway, Wnt/0-catenin pathway, intrinsic (mitochondrial) pathway, extrinsic (death receptor) pathway, caspase cascade, Notch signaling pathway, hedgehog signaling pathway, TGF-β (transforming growth factor Beta) pathway, JAK/STAT pathway, G-protein coupled receptor (GPCR) pathway, calcium signaling pathway, glycolysis, citric acid cycle (Krebs Cycle), oxidative phosphorylation, lipid metabolism pathway, amino acid metabolism, Toll-like receptor (TLR) pathway, NF-xB signaling pathway, complement pathway, nucleotide excision repair (NER), base excision repair (BER), mismatch repair (MMR), cyclin-dependent kinase (CDK) pathway, Rb (retinoblastoma) pathway, p53 pathway, unfolded protein response (UPR), heat shock response pathway, oxidative stress pathway, BMP (bone morphogenetic protein) pathway, FGF (fibroblast growth factor) pathway, Sonic Hedgehog pathway, neurotrophin signaling pathway, synaptic transmission pathway, axon guidance pathways, insulin signaling pathway, thyroid hormone pathway, steroid hormone pathway, VEGF (vascular endothelial growth factor) pathway, DNA methylation pathway, histone modification pathway, or angiogenesis. In embodiments, the biomolecule specific binding agent is capable of binding to a biomolecule on the surface of or in a B cell, Mature B Cell, Follicular B cell, Marginal Zone B cell, Short lived plasma cell, Memory B cell, Long lived plasma cell, B1 cell, Breg, Germinal Center B cell, Macrophage, Monocyte, M1 macrophage, M2 macrophage, Dendritic Cell, Plasmacytoid dendritic cell, Monocyte-derived dendritic cell, T cell, T Follicular Helper, Th1, Th2, Th9, Th17, Th22, Treg, platelet (activated), platelet (rested), natural killer cell, neutrophil, basophil, eosinophil, mast cell, astrocyte, neuron, glial cell, lymphocyte, myeloid cell, granulocytes, neural cells, stem cells, endothelial cells, epithelial cells, mesenchymal stem cell, hematopoietic stem cell, embryonic stem, stromal cell, erythrocyte, fibroblast, or apoptotic cell.

In embodiments, the specific binding agent is a monoclonal antibody or a polyclonal antibody. In embodiments, the specific binding agent is capable of binding (e.g., capable of specifically binding) to an actin filament of a cell, a plasma membrane of a cell, a mitochondria of a cell, the endoplasmic reticulum of a cell, a tubule of the endoplasmic reticulum, a cisternae of the endoplasmic reticulum, sheets and tubules of the endoplasmic reticulum, a nuclear envelope of the endoplasmic reticulum, a Golgi apparatus of a cell, cisternae of the Golgi apparatus, a lysosome of a cell, phosphatidylserine, a cell surface carbohydrate, or a transferrin receptor. In embodiments, the specific binding agent is capable of binding a carbohydrate on a cell surface. In embodiments, the specific binding agent is capable of binding a glycolipid, a glycoprotein, an α-glucopyranosyl residue on a cell membrane, an N-acetylglucosaminyl residue on a cell membrane, an N-acetylneuraminic acid (sialic acid) on a cell membrane, peroxisome, a nucleus, an endosome, or a cytoskeletal protein. In embodiments, the cytoskeletal protein includes talin. In embodiments, the cytoskeletal protein includes tubulin. In embodiments, the specific binding agent is a monovalent phalloidin molecule, monovalent wheat germ agglutinin molecule, monovalent concanavalin A molecule, an annexin molecule, transferrin molecule, lectin molecule, or Hoescht 33342. In embodiments, the specific binding agent is a cell paint (see, e.g., Gustafsdottir S. M. et al. PLoS One. 2013 Dec. 2; 8(12):e80999).

Specific antibodies tagged with known oligonucleotide sequences can be synthesized by using bifunctional crosslinkers reactive towards thiol (via maleimide) and amine (via NHS) moieties. For example, a 5′-thiol-modified oligonucleotide could be conjugated to a crosslinker via maleimide chemistry and purified. The oligos with a 5′-NHS-ester would then be added to a solution of antibodies and reacted with amine residues on the antibodies surface to generate tagged antibodies capable of binding analytes with target epitopes. These tagged antibodies include oligonucleotide sequence(s). The one or more oligonucleotide sequences may include a barcode, binding sequences (e.g., primer binding sequence or sequences complementary to hybridization pads), and/or unique molecular identifier (UMI) sequences.

In embodiments, specific binding entails a binding affinity, expressed as a K_{D} (such as a K_{D} measured by surface plasmon resonance at an appropriate temperature, such as 37° C.). In embodiments, the K_{D} of a specific binding interaction is less than about 100 nM, 50 nM, 10 nM, 1 nM, 0.05 nM, or lower. In embodiments, the K_{D} of a specific binding interaction is about 0.01-100 nM, 0.1-50 nM, or 1-10 nM. In embodiments, the K_{D} of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically, a specific or selective reaction will be at least twice background signal to noise and more typically more than 10 to 100 times greater than background.

In embodiments, prior to step (i), the method includes forming the first circular polynucleotide by hybridizing a first end and a second end of a single-stranded polynucleotide (i.e., a circularizable oligonucleotide) to the first nucleic acid molecule and ligating the first end and second end together to form the first circular polynucleotide. For example, the first end includes a first hybridization sequence and the second end includes a second hybridization sequence, wherein each hybridization sequence is complementary to a respective sequence of the nucleic acid molecule.

In embodiments, prior to step (i), the method includes forming the second circular polynucleotide by hybridizing a first end and a second end of a single-stranded polynucleotide (i.e., a circularizable oligonucleotide) to the second nucleic acid molecule and ligating the first end and second end together to form the second circular polynucleotide. For example, the first end includes a first hybridization sequence and the second end includes a second hybridization sequence, wherein each hybridization sequence is complementary to a respective sequence of the nucleic acid molecule.

In embodiments, hybridizing a first end and a second end of a single-stranded polynucleotide described herein to a nucleic acid molecule described herein includes incubation in a buffer at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. In embodiments, hybridizing a first end and a second end of a single-stranded polynucleotide described herein to a nucleic acid molecule described herein includes incubation in a buffer at 40° C. to 50° C. In embodiments, hybridizing a first end and a second end of a single-stranded polynucleotide described herein to a nucleic acid molecule described herein includes incubation in a buffer at 50° C. to 60° C.

In embodiments, ligating includes incubation in a buffer at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. In embodiments, ligating includes incubation in a buffer 30° C. to 40° C. In embodiments, ligating includes incubation in a buffer 40° C. to 50° C.

In embodiments, prior to step (i), the method includes forming the first circular polynucleotide by hybridizing a first end and a second end of a single-stranded polynucleotide (i.e., a circularizable oligonucleotide) to the first nucleic acid molecule, extending the first end along the first nucleic acid molecule, and ligating the first end and the extended second end together to form the first circular polynucleotide. For example, the first end includes a first hybridization sequence and the second end includes a second hybridization sequence, wherein each hybridization sequence is complementary to a respective sequence of the nucleic acid molecule which flanks one or more nucleotides (e.g., a gap).

In embodiments, prior to step (i), the method includes forming the second circular polynucleotide by hybridizing a first end and a second end of a single-stranded polynucleotide (i.e., a circularizable oligonucleotide) to the second nucleic acid molecule, extending the second end along the second nucleic acid molecule, and ligating the first end and the extended second end together to form the second circular polynucleotide. For example, the first end includes a first hybridization sequence and the second end includes a second hybridization sequence, wherein each hybridization sequence is complementary to a respective sequence of the nucleic acid molecule which flanks one or more nucleotides (e.g., a gap). In embodiments, extending the second end (e.g., a 3′ end of the single-stranded polynucleotide described herein) along the nucleic acid described herein includes incubation in a buffer at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. In embodiments, extending the second end (e.g., a 3′ end of the single-stranded polynucleotide described herein) along the nucleic acid described herein includes incubation in a buffer 30° C. to 40° C.

In embodiments, the method includes circularizing the single-stranded polynucleotide described herein to form a circular polynucleotide described herein. In embodiments, the circularizing includes intramolecular joining of the 5′ and 3′ ends of a linear nucleic acid molecule (e.g., the single-stranded polynucleotide described herein). In embodiments, the circularizing includes a ligation reaction. In embodiments, the two ends of the linear nucleic acid molecule are ligated directly together. In embodiments, the two ends of the linear nucleic acid molecule are ligated together with the aid of a bridging oligonucleotide (sometimes referred to as a splint oligonucleotide) that is complementary with the two ends of the linear nucleic acid molecule. Methods for forming circular DNA templates are known in the art, for example, linear polynucleotides are circularized in a non-template driven reaction with circularizing ligase, such as CircLigase™, CircLigase™ II, Taq DNA Ligase, HiFi Taq DNA Ligase, T4 DNA ligase, or Ampligase® DNA Ligase. In some embodiments, circularization is facilitated by denaturing double-stranded linear nucleic acids prior to circularization. Residual linear DNA molecules may be optionally digested. In some embodiments, circularization is facilitated by chemical ligation (e.g., click chemistry, e.g., a copper-catalyzed reaction of an alkyne (e.g., a 3′ alkyne) and an azide (e.g., a 5′ azide)). In embodiments, prior to circularization, the linear DNA fragments are A-tailed (e.g., A-tailed using Taq DNA polymerase). In embodiments, circularization of the linear nucleic acid molecule is performed with CircLigase™ enzyme. In embodiments, circularization of the linear nucleic acid molecule is performed with a thermostable RNA ligase, or mutant thereof. In embodiments, circularization of the linear nucleic acid molecule is performed with an RNA ligase enzyme from bacteriophage TS2126, or mutant thereof. For example, the RNA ligase may be TS2126 RNA ligase, as described in U.S. Pat. Pub. 2005/0266439, which is incorporated herein by reference in its entirety. In embodiments, circularizing includes ligating a first hairpin and a second hairpin adapter to a linear nucleic acid molecule, thereby forming a circular polynucleotide.

In embodiments, the circularizable oligonucleotide is a single-stranded polynucleotide described herein. In embodiments, the circularizable oligonucleotide includes a primer binding sequence. In embodiments, the circularizable oligonucleotide includes at least one primer binding sequence. In embodiments, the circularizable oligonucleotide includes at least two primer binding sequences. In embodiments, the circularizable oligonucleotide includes a primer binding sequence from a known set of primer binding sequences. In embodiments, the circularizable oligonucleotide includes at least two primer binding sequences from a known set of primer binding sequences. In embodiments, the circularizable oligonucleotide includes up to 50 different primer binding sequences from a known set of primer binding sequences. In embodiments, the circularizable oligonucleotide includes up to 10 different primer binding sequences from a known set of primer binding sequences. In embodiments, the circularizable oligonucleotide includes up to 5 different primer binding sequences from a known set of primer binding sequences. In embodiments, the circularizable oligonucleotide includes two or more sequencing primer binding sequences from a known set of sequencing primer binding sequences. In embodiments the circularizable oligonucleotide includes 1, 2, 3, 4, 5, 6, 7, 8, 9, or 10 primer binding sequences from a known set of primer binding sequences. In embodiments, the circularizable oligonucleotide includes two or more different primer binding sequences from a known set of primer binding sequences. In embodiments, the circularizable oligonucleotide includes 1, 2, 3, 4, 5, 6, 7, 8, 9, or 10 different primer binding sequences from a known set of primer binding sequences. In embodiments, the circularizable oligonucleotide includes 2 to 5 primer binding sequences from a known set of primer binding sequences. In embodiments, the circularizable oligonucleotide includes 2 to 5 different primer binding sequences from a known set of primer binding sequences. In embodiments, the circularizable oligonucleotide includes 2 to 5 sequencing primer binding sequences from a known set of sequencing primer binding sequences. In embodiments, the circularizable oligonucleotide includes 2 to 5 different sequencing primer binding sequences from a known set of sequencing primer binding sequences. In embodiments, the circularizable oligonucleotide includes at least two different primer binding sequences. In embodiments, the circularizable oligonucleotide includes two different sequencing primer binding sequences.

In embodiments, the circularizable oligonucleotide includes about 50 to about 150 nucleotides. In embodiments, the circularizable oligonucleotide includes about 50 to about 300 nucleotides. In embodiments, the circularizable oligonucleotide includes about 50 to about 500 nucleotides. In embodiments, the circularizable oligonucleotide includes about or more than about 50, 75, 100, 125, 150, 175, 200, 250, 300, 350, 400, or 500 nucleotides. In embodiments, the circularizable oligonucleotide includes less than about 50, 75, 100, 125, 150, 175, 200, 250, 300, 350, 400, or 500 nucleotides.

In embodiments, the first circular polynucleotide and the second circular polynucleotide are each about 50 to about 500 nucleotides. In embodiments, the first circular polynucleotide and the second circular polynucleotide are each about 70 to about 150 nucleotides. In embodiments, the first circular polynucleotide and the second circular polynucleotide are substantially similar in size. In embodiments, the first circular polynucleotide includes more nucleotides than the second circular polynucleotide. In embodiments, the first circular polynucleotide and the second circular polynucleotide are each about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 nucleotides.

In embodiments, the circular oligonucleotide includes about 50 to about 150 nucleotides. In embodiments, the circular oligonucleotide includes about 50 to about 300 nucleotides. In embodiments, the circular oligonucleotide includes about 50 to about 500 nucleotides. In embodiments, the circular oligonucleotide includes about or more than about 50, 75, 100, 125, 150, 175, 200, 250, 300, 350, 400, or 500 nucleotides. In embodiments, the circular oligonucleotide includes less than about 50, 75, 100, 125, 150, 175, 200, 250, 300, 350, 400, or 500 nucleotides.

In embodiments, the first nucleic acid molecule is an RNA molecule. In embodiments, the first nucleic acid molecule is a DNA molecule. In embodiments, the second nucleic acid molecule is an RNA molecule. In embodiments, the second nucleic acid molecule is a DNA molecule. In embodiments, the nucleic acid molecule is referred to as a target polynucleotide. In embodiments, the target polynucleotide is an RNA nucleic acid sequence or DNA nucleic acid sequence. In embodiments, the target polynucleotide is an RNA nucleic acid sequence or DNA nucleic acid sequence from the same cell. In embodiments, the target polynucleotide is an RNA nucleic acid sequence. In embodiments, the RNA nucleic acid sequence is stabilized using known techniques in the art. For example, RNA degradation by RNase should be minimized using commercially available solutions, e.g., RNA Later®, RNA Lysis Buffer, or Keratinocyte serum-free medium). In embodiments, the target polynucleotide is messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target polynucleotide is pre-mRNA. In embodiments, the target polynucleotide is heterogeneous nuclear RNA (hnRNA). In embodiments, the target polynucleotide is mRNA, tRNA (transfer RNA), rRNA (ribosomal RNA), or noncoding RNA (such as lncRNA (long noncoding RNA)). In embodiments, the target polynucleotides are on different regions of the same RNA nucleic acid sequence.

In embodiments, the target polynucleotide includes RNA nucleic acid sequences. In embodiments the target polynucleotide is an RNA transcript. In embodiments the target polynucleotide is a single stranded RNA nucleic acid sequence. In embodiments, the target polynucleotide is an RNA nucleic acid sequence or a DNA nucleic acid sequence (e.g., cDNA). In embodiments, the target polynucleotide is a cDNA target polynucleotide nucleic acid sequence and before step a), the RNA nucleic acid sequence is reverse transcribed to generate the cDNA target polynucleotide nucleic acid sequence. In embodiments, reverse transcription of the RNA nucleic acid is performed with a reverse transcriptase, for example, Tth DNA polymerase or mutants thereof. In embodiments, the target polynucleotide is genomic DNA (gDNA), mitochondrial DNA, chloroplast DNA, episomal DNA, viral DNA, or copy DNA (cDNA). In embodiments, the target polynucleotide is coding RNA such as messenger RNA (mRNA), and non-coding RNA (ncRNA) such as transfer RNA (tRNA), microRNA (miRNA), small nuclear RNA (snRNA), or ribosomal RNA (rRNA). In embodiments, the target polynucleotide is a cancer-associated gene. In embodiments, to minimize amplification errors or bias, the target polynucleotide is not reverse transcribed to generate cDNA.

In embodiments, the second number is about 1%, about 5%, about 10%, about 15%, about 20%, about 25%, about 30%, about 40%, about 50%, about 75% more than the first number. In embodiments, the second number is about 2-fold, at least about 1.5-fold, at least about 2.0-fold, at least about 2.5-fold, at least about 5-fold, at least about 10-fold, or more than about 10-fold greater than the first number. In embodiments, the second number is about 1.0-fold greater than the first number. In embodiments, the second number is about 2.0-fold greater than the first number. In embodiments, the second number is about 5.0-fold greater than the first number. In embodiments, the second number is about 20-fold greater than the first number. In embodiments, the second number is about 2-fold, at least about 1.5-fold, at least about 2.0-fold, at least about 2.5-fold, at least about 5-fold, at least about 10-fold, or more than about 10-fold than the first number. In embodiments, the second number of copies of the second sequence is about 1%, about 5%, about 10%, about 15%, about 20%, about 25%, about 30%, about 40%, about 50%, about 75% more than the first number of copies of the first sequence. In embodiments, the second number of copies of the second sequence is about 2-fold, at least about 1.5-fold, at least about 2.0-fold, at least about 2.5-fold, at least about 5-fold, at least about 10-fold, or more than about 10-fold than the first number of copies of the first sequence.

In embodiments, the second number is about 1%, about 5%, about 10%, about 15%, about 20%, about 25%, about 30%, about 40%, about 50%, about 75% more than the first number. In embodiments, the second number is about 0.01%, about 0.05%, about 0.010%, about 0.015%, about 0.020%, about 0.025%, about 0.030%, about 0.040%, about 0.050%, about 0.075% more than the first number. In embodiments, the second number is about 0.1%, about 0.5%, about 0.10%, about 0.15%, about 0.20%, about 0.25%, about 0.30%, about 0.40%, about 0.50%, about 0.75% more than the first number. In embodiments, the second number is greater than the first number. In embodiments, the first number is about 1%, about 5%, about 10%, about 15%, about 20%, about 25%, about 30%, about 40%, about 50%, about 75% less than the second number. In embodiments, the first number is about 0.01%, about 0.05%, about 0.010%, about 0.015%, about 0.020%, about 0.025%, about 0.030%, about 0.040%, about 0.050%, about 0.075% less than the second number. In embodiments, the first number is about 0.1%, about 0.5%, about 0.10%, about 0.15%, about 0.20%, about 0.25%, about 0.30%, about 0.40%, about 0.50%, about 0.75% less than the second number. In embodiments, the second number quantified after 1, 2, 3, 4, 5, 10, 15, or 20 minutes of amplification (e.g., eRCA or RCA) is measurably higher than the first number. In embodiments, the second number quantified after 6, 7, 8, 9, 10, 11, 12, or 16 hours of amplification (e.g., RCA) is measurably higher than the first number.

In embodiments, the second number quantified after one cycle of extension is measurably higher than the first number. In embodiments, the method generates a first number of non-fusion polynucleotide amplification products and a second number of fusion polynucleotide amplification products at a ratio of 1.00:1.01. In embodiments, the ratio of first number to second number is 1.00:1.02. In embodiments, the ratio of first number to second number is 1.00:1.05. In embodiments, the ratio of first number to second number is 1.00:1.10.

In embodiments, amplifying the first circular polynucleotide includes extending the first nucleic acid molecule. For example, the first nucleic acid molecule is covalently attached to an antibody, and when hybridized to a circular polynucleotide it may serve as an amplification primer for initiating a rolling circle amplification process. In this manner, the amplification product is thus covalently attached to the antibody, wherein the antibody is bound to the protein of interest, which may aid in localizing the amplification product. Alternatively, a new primer may be introduced. For example, in embodiments, amplifying the first circular polynucleotide includes hybridizing a first amplification primer to the first circular polynucleotide and extending the first amplification primer.

In embodiments, amplifying the second circular polynucleotide includes hybridizing a second amplification primer to the second circular polynucleotide and extending the second amplification primer.

In embodiments, during amplification (e.g., after about 15 minutes of amplification) streptavidin contacts and binds to the retarding agent on the first circular polynucleotide, wherein the retarding agent is a biotin-nucleotide, thereby effectively arresting amplification for the first circular polynucleotide. In embodiments, during amplification (e.g., after about 15 minutes of amplification) streptavidin contacts and binds to the biotin-nucleotide, effectively arresting amplification for the circular oligonucleotide (e.g., a circular polynucleotide described herein). In embodiments, the method includes reducing or eliminating amplification for a portion of a plurality of amplification reactions.

In embodiments, amplifying the circular oligonucleotide (e.g., a circular polynucleotide described herein) includes incubating the circular oligonucleotide with the strand-displacing polymerase for about 1 hour to about 12 hours. In embodiments, amplifying includes incubation with the strand-displacing polymerase for about 60 seconds to about 60 minutes. In embodiments, amplifying includes incubation with the strand-displacing polymerase for about 10 minutes to about 60 minutes. In embodiments, amplifying includes incubation with the strand-displacing polymerase for about 10 minutes to about 30 minutes. In embodiments, amplifying the circular oligonucleotide (e.g., a circular polynucleotide described herein) includes incubating the circular oligonucleotide with the strand-displacing polymerase for about 1, about 2, about 3, about 4, about 5, about 6, about 7, about 8, about 9, about 10, about 11, or about 12 hours. In embodiments, amplifying the circular oligonucleotide (e.g., a circular polynucleotide described herein) includes incubating the circular oligonucleotide with the strand-displacing polymerase for more than 12 hours. In embodiments, amplifying includes incubation with the strand-displacing polymerase for about 15 minutes to about 60 minutes.

In embodiments, amplifying the circular oligonucleotide (e.g., a circular polynucleotide described herein) includes incubating the circular oligonucleotide with the strand-displacing polymerase at a temperature of about 20° C. to about 50° C. In embodiments, incubation with the strand-displacing polymerase is at a temperature of about 20° C., about 25° C., about 30° C., about 35° C., about 40° C., about 45° C., or about 50° C. In embodiments, incubation with the strand-displacing polymerase is at a temperature of about 35° C. to 42° C. In embodiments, incubation with the strand-displacing polymerase is at a temperature of about 35° C., about 36° C., about 37° C., about 38° C., about 39° C., about 40° C., about 41° C., or about 42° C. In embodiments, the strand-displacing polymerase is a phi29 polymerase, a SD polymerase, a Bst large fragment polymerase, phi29 mutant polymerase, a Thermus aquaticus polymerase, or a thermostable phi29 mutant polymerase.

In embodiments, amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is Bst DNA Polymerase Large Fragment, Thermus aquaticus (Taq) polymerase, or a mutant thereof. In embodiments, the strand-displacing polymerase is a phi29 polymerase, a phi29 mutant polymerase or a thermostable phi29 mutant polymerase. A “phi polymerase” (or “Φ29 polymerase”) is a DNA polymerase from the Φ29 phage or from one of the related phages that, like Φ29, contain a terminal protein used in the initiation of DNA replication. For example, phi29 polymerases include the B103, GA-1, PZA, Φ15, BS32, M2Y (also known as M2), Nf, G1, Cp-1, PRD1, PZE, SFS, Cp-5, Cp-7, PR4, PR5, PR722, L17, Φ21, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase read length, enhance accuracy, increase phototolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes.

In embodiments, the amplification method includes a standard dNTP mixture including dATP, dCTP, dGTP and dTTP (for DNA) or dATP, dCTP, dGTP and dUTP (for RNA). In embodiments, the amplification method includes a mixture of standard dNTPs and modified nucleotides that contain functional moieties (e.g., bioconjugate reactive groups) that serve as attachment points to the cell or the matrix in which the cell is embedded (e.g. a hydrogel). In embodiments, the amplification method includes a mixture of standard dNTPs and modified nucleotides that contain functional moieties (e.g., bioconjugate reactive groups) that participate in the formation of a bioconjugate linker. The modified nucleotides may react and link the amplification product to the surrounding cell scaffold. For example, amplifying may include an extension reaction wherein the polymerase incorporates a modified nucleotide into the amplification product, wherein the modified nucleotide includes a bioconjugate reactive moiety (e.g., an alkynyl moiety) attached to the nucleobase. The bioconjugate reactive moiety of the modified nucleotide participates in the formation of a bioconjugate linker by reacting with a complementary bioconjugate reactive moiety present in the cell (e.g., a crosslinking agent, such as NHS-PEG-azide, or an amine moiety) thereby attaching the amplification product to the internal scaffold of the cell. In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)₉)).

In embodiments, the method further includes detecting the amplification products. In embodiments, detecting includes binding a detection agent (e.g., a labeled probe) to the amplification product. In embodiments, the detection agent includes a fluorescently labeled probe. In embodiments, the method includes exciting and detecting the label. In embodiments, detecting includes serially contacting the amplification products with labeled probes (e.g., labeled oligonucleotides or labeled nucleotides). In embodiments, detecting includes sequencing.

The phrase “labeled probes” refers to mixture of nucleic acids that are detectably labeled, e.g., fluorescently labeled, such that the presence of the probe, as well as, any target sequence to which the probe is bound can be detected by assessing the presence of the label. In some embodiments, the probes are about 30-300 bases in length, 40-300 bases in length, or 70-300 bases in length. In some embodiments, the probes are relatively uniform in length (e.g., an average length+/−10 bases). The probes may be uniformly labeled based on position of label and/or number of labels within the probe. In some embodiments, the probes are single-stranded. In some embodiments, the probes are double-stranded. Additional detection probes and related properties may be found in, e.g., U.S. Pat. Pub. US 2011/0039735, which is incorporated herein by reference in its entirety. In embodiments, the method includes hybridizing a primer to the amplification product and incorporating a labeled nucleotide into the primer.

In embodiments, sequencing includes hybridizing a sequencing primer to the amplification product and incorporating one or more labeled nucleotides, and detecting the incorporated one or more labeled nucleotides so as to identify the sequence.

In embodiments, the method includes sequencing the amplification products. In embodiments, sequencing includes a plurality of sequencing cycles. In embodiments, sequencing includes a plurality of rounds of sequencing cycles (e.g., a first round of 10 sequencing cycles; followed by a second round of 10 sequencing cycles). In embodiments, sequencing includes a plurality of rounds of sequencing cycles (e.g., a first round of 1 sequencing cycle; followed by a second round of 1 sequencing cycle). In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, prior to initiating a next round of sequencing cycles, the first sequencing primer is terminated or removed. For example, termination may occur via incorporating a non-extendable nucleotide (e.g., a ddNTP) into the first sequencing primer.

In embodiments, the method includes sequencing the barcode (e.g., the barcode sequence or the barcode nucleotide). In embodiments, the method includes sequencing a plurality of barcodes in an optically resolved volume. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, are subjected to repeated cycles of oligonucleotide delivery and detection. SBL methods, include those described in Shendure et al. Science 309:1728-1732 (2005); U.S. Pat. Nos. 5,599,675; and 5,750,341, each of which are incorporated herein by reference in their entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which are incorporated herein by reference in their entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. In embodiments, sequencing includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, sequencing may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. By ordering the products derived using these modified nucleotides it is possible to deduce the DNA sequence of the oligonucleotide barcode.

In embodiments, sequencing includes extending a first sequencing primer to generate a sequencing read including the first barcode sequence, or a portion thereof. In embodiments, sequencing includes extending a first sequencing primer to generate a sequencing read including the first barcode sequence, or a portion thereof, and extending a second sequencing primer to generate a sequencing read including the second barcode sequence. In embodiments, sequencing includes sequentially extending a plurality of sequencing primers (e.g., sequencing a first region of a target nucleic acid followed by sequencing a second region of a target nucleic acid, followed by sequencing N regions, where N is the number of sequencing primers in the known sequencing primer set). In embodiments, sequencing includes generating a plurality of sequencing reads.

In embodiments, sequencing includes sequentially sequencing a plurality of different targets by initiating sequencing with different sequencing primers. For example, a first circularizable probe includes a first primer binding site (a nucleic acid sequence complementary to a first sequencing primer) and optionally a first barcode sequence or barcode nucleotide. In a similar manner, a second and third padlock probe include a second primer binding site (a nucleic acid sequence complementary to a second, different, sequencing primer) and a third primer binding site (a nucleic acid sequence complementary to a third, different from both Primer 1 and Primer 2, sequencing primer), respectively. During the first round of sequencing (following probe circularization and amplification according to the methods described herein), using primer 1, the probe hybridized to the first nucleic acid molecule is detected. In the second round of sequencing, primer 2 can hybridize and sequence an identifying sequence of the probe (e.g., a barcode sequence or nucleotide) hybridized to a second nucleic acid molecule. Similarly, in the third round of sequencing, primer 3 can hybridize and sequence the probe hybridized to the third nucleic acid molecule.

In embodiments, sequencing includes encoding the sequencing read into a codeword. Useful encoding schemes include those developed for telecommunications, coding theory and information theory such as those set forth in Hamming, Coding and Information Theory, 2^{nd} Ed. Prentice Hall, Englewood Cliffs, N.J. (1986) and Moon TK. Error Correction Coding: Mathematical Methods and Algorithms. ed. 1st Wiley: 2005., each of which are incorporated herein by reference. A useful encoding scheme uses a Hamming code. A Hamming code can provide for signal (and therefore sequencing and barcode) distinction. In this scheme, signal states detected from a series of nucleotide incorporation and detection events (i.e., while sequencing the oligonucleotide barcode) can be represented as a series of the digits to form a codeword, the codeword having a length equivalent to the number incorporation/detection events. The digits can be binary (e.g. having a value of 1 for presence of signal and a value of 0 for absence of the signal) or digits can have a higher radix (e.g., a ternary digit having a value of 1 for fluorescence at a first wavelength, a value of 2 for fluorescence at a second wavelength, and a value of 0 for no fluorescence at those wavelengths, etc.). Barcode discrimination capabilities are provided when codewords can be quantified via Hamming distances between two codewords (i.e., barcode 1 having codeword 1, and barcode 2 having codeword 2, etc.).

In embodiments, sequencing includes extending a sequencing primer to generate a sequencing read. In embodiments, sequencing includes extending a sequencing primer by incorporating a labeled nucleotide, or labeled nucleotide analogue and detecting the label to generate a signal for each incorporated nucleotide or nucleotide analogue. In embodiments, the labeled nucleotide or labeled nucleotide analogue further includes a reversible terminator moiety.

In embodiments, the labeled nucleotide or labeled nucleotide analogue further includes a reversible terminator moiety. In embodiments, the reversible terminator moiety is attached to the 3′ oxygen of the nucleotide and is independently

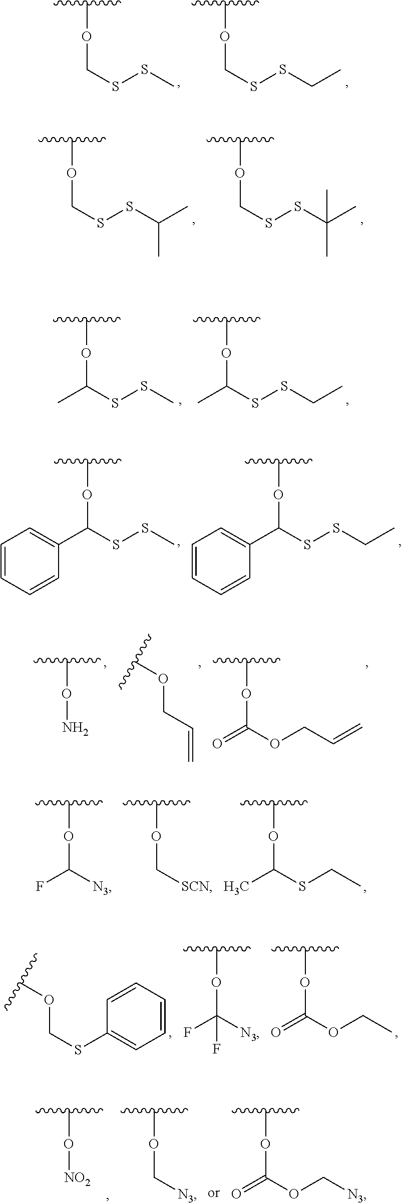

wherein the 3′ oxygen is explicitly depicted in the above formulae. In embodiments, the reversible terminator moiety is attached to the 3′ oxygen of the nucleotide and is an allyl moiety. Additional examples of reversible terminators may be found in U.S. Pat. No. 6,664,079, Ju J. et al. (2006) Proc Natl Acad Sci USA 103(52):19635-19640; Ruparel H. et al. (2005) Proc Natl Acad Sci USA 102(17):5932-5937; Wu J. et al. (2007) Proc Natl Acad Sci USA 104(104):16462-16467; Guo J. et al. (2008) Proc Natl Acad Sci USA 105(27): 9145-9150 Bentley D. R. et al. (2008) Nature 456(7218):53-59; or Hutter D. et al. (2010) Nucleosides Nucleotides & Nucleic Acids 29:879-895, which are incorporated herein by reference in their entirety for all purposes. In embodiments, a polymerase-compatible cleavable moiety includes an azido moiety or a dithiol moiety.

A variety of suitable sequencing platforms are available for implementing methods disclosed herein (e.g., for performing the sequencing reaction). Non-limiting examples include SMRT (single-molecule real-time sequencing), ion semiconductor, pyrosequencing, sequencing by synthesis, sequencing by binding, combinatorial probe anchor synthesis, SOLiD sequencing (sequencing by ligation), and nanopore sequencing. Sequencing platforms include those provided by Singular Genomics™ (e.g., the G4™ system) or Jllumina™, Inc. (e.g., HiSeq™, MiSeq™, NextSeq™, or NovaSeq™ systems).

In embodiments, generating a sequencing read includes determining the identity of the nucleotides in the template polynucleotide (or complement thereof). In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides. In embodiments, following the generation of a sequencing read, subsequent extension is performed using a plurality of standard (e.g., non-modified) dNTPs until the complementary strand is copied. In other embodiments, following the generation of a sequencing read, subsequent extension is performed using a plurality of dideoxy nucleotide triphosphates (ddNTPs) to prevent further extension of the first sequencing read product during a second sequencing read. In embodiments, following the identification of at least 5-10 (e.g., 11 to 200 nucleotides, or up to 1000 nucleotides), subsequent extension is performed using a plurality of standard (e.g., non-modified) dNTPs until the complementary strand is copied. In embodiments, following the identification of at least 5-10 (e.g., 11 to 200 nucleotides, or up to 1000 nucleotides), subsequent extension is performed using a plurality of dideoxy nucleotide triphosphates (ddNTPs) to prevent further extension of the sequencing read product.

In embodiments, the methods of sequencing a nucleic acid include extending a complementary polynucleotide (e.g., a primer) that is hybridized to the nucleic acid by incorporating a first nucleotide. In embodiments, the method includes a buffer exchange or wash step. In embodiments, the methods of sequencing a nucleic acid include a sequencing solution. The sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof; and (d) a guanine nucleotide, or analog thereof.

In embodiments, the method includes sequencing a plurality of target polynucleotides of a cell in situ within an optically resolved volume. In embodiments, the number of unique targets detected within an optically resolved volume of a sample is about 3, 10, 30, 50, or 100. In embodiments, the number of unique targets detected within an optically resolved volume of a sample is about 1 to 10. In embodiments, the number of unique targets detected within an optically resolved volume of a sample is about 5 to 10. In embodiments, the number of unique targets detected within an optically resolved volume of a sample is about 1 to 5. In embodiments, the number of unique targets detected within an optically resolved volume of a sample is at least 3, 10, 30, 50, or 100. In embodiments, the number of unique targets detected within an optically resolved volume of a sample is less than 3, 10, 30, 50, or 100. In embodiments, the number of unique targets detected within an optically resolved volume of a sample is about 1, 2, 3, 4, 5, 6, 7, 8, 9, 10, 20, 50, 100, 500, 1,000, 5,000, 10,000, or 200,000. In embodiments, the methods allow for detection of a single target of interest. In embodiments, the methods allow for multiplex detection of a plurality of targets of interest.

In embodiments, the optically resolved volume has an axial resolution (i.e., depth, or z) that is greater than the lateral resolution (i.e., xy plane). In embodiments, the optically resolved volume has an axial resolution that is greater than twice the lateral resolution. In embodiments, the dimensions (i.e., the x, y, and z dimensions) of the optically resolved volume are about 0.5 μm×0.5 μm×0.5 μm; 1 μm×1 μm×1 μm; 2 μm×2 μm×2 μm; 0.5 μm×0.5 μm×1 μm; 0.5 μm×0.5 μm×2 μm; 2 μm×2 μm×1 μm; or 1 μm×1 μm×2 μm. In embodiments, the dimensions (i.e., the x, y, and z dimensions) of the optically resolved volume are about 1 μm×1 μm×2 μm; 1 μm×1 μm×3 μm; 1 μm×1 μm×4 μm; or about 1 μm×1 μm×5 μm. In embodiments, the dimensions (i.e., the x, y, and z dimensions) of the optically resolved volume are about 1 μm×1 μm×5 μm. In embodiments, the dimensions (i.e., the x, y, and z dimensions) of the optically resolved volume are about 1 μm×1 μm×6 μm. In embodiments, the dimensions (i.e., the x, y, and z dimensions) of the optically resolved volume are about 1 μm×1 μm×7 μm. In embodiments, the optically resolved volume is a cubic micron. In embodiments, the optically resolved volume has a lateral resolution from about 100 to 200 nanometers, from 200 to 300 nanometers, from 300 to 400 nanometers, from 400 to 500 nanometers, from 500 to 600 nanometers, or from 600 to 1000 nanometers. In embodiments, the optically resolved volume has a axial resolution from about 100 to 200 nanometers, from 200 to 300 nanometers, from 300 to 400 nanometers, from 400 to 500 nanometers, from 500 to 600 nanometers, or from 600 to 1000 nanometers. In embodiments, the optically resolved volume has a axial resolution from about 1 to 2 μm, from 2 to 3 μm, from 3 to 4 μm, from 4 to 5 μm, from 5 to 6 μm, or from 6 to 10 μm.

In embodiments, the method further includes measuring an amount of one or more of the targets by counting the one or more associated sequences. In embodiments, the method further includes counting the one or more associated sequences in an optically resolved volume.

In embodiments, the number of unique targets detected within an optically resolved volume of a sample is about 3, 10, 30, 50, or 100. In embodiments, the number of unique targets detected within an optically resolved volume of a sample is about 1 to 10. In embodiments, the number of unique targets detected within an optically resolved volume of a sample is about 5 to 10. In embodiments, the number of unique targets detected within an optically resolved volume of a sample is about 1 to 5. In embodiments, the number of unique targets detected within an optically resolved volume of a sample is at least 3, 10, 30, 50, or 100. In embodiments, the number of unique targets detected within an optically resolved volume of a sample is less than 3, 10, 30, 50, or 100. In embodiments, the number of unique targets detected within an optically resolved volume of a sample is about 1, 2, 3, 4, 5, 6, 7, 8, 9, 10, 20, 50, 100, 500, 1,000, 5,000, 10,000, or 200,000. In embodiments, the methods allow for detection of a single target of interest. In embodiments, the methods allow for multiplex detection of a plurality of targets of interest. The use of oligonucleotide barcodes with unique identifier sequences as described herein allows for simultaneous detection of 2, 3, 4, 5, 6, 7, 8, 9, 10, 20, 30, 40, 50, 60, 70, 80, 90, 100, 200, 300, 400, 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000 or more than 10,000 unique targets within a single cell. In contrast to existing in situ detection methods, the methods presented herein have the advantage of virtually limitless numbers of individually detected molecules in parallel and in situ.

In embodiments, the method further includes an additional imaging modality, immunofluorescence (IF), or immunohistochemistry modality (e.g., immunostaining). In embodiments, the method includes ER staining (e.g., contacting the cell with a cell-permeable dye which localizes to the endoplasmic reticula), Golgi staining (e.g., contacting the cell with a cell-permeable dye which localizes to the Golgi), F-actin staining (e.g., contacting the cell with a phalloidin-conjugated dye that binds to actin filaments), lysosomal staining (e.g., contacting the cell with a cell-permeable dye that accumulates in the lysosome via the lysosome pH gradient), mitochondrial staining (e.g., contacting the cell with a cell-permeable dye which localizes to the mitochondria), nucleolar staining, or plasma membrane staining. For example, the method includes live cell imaging (e.g., obtaining images of the cell) prior to or during fixing, immobilizing, and permeabilizing the cell. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)).

In embodiments, the method further includes detecting the amplification product (e.g., the amplification product of step (d)). In embodiments, detecting includes two-dimensional (2D) or three-dimensional (3D) fluorescent microscopy. Suitable imaging technologies are known in the art, as exemplified by Larsson et al., Nat. Methods (2010) 7:395-397 and associated supplemental materials, the entire content of which is incorporated by reference herein in its entirety. In embodiments of the methods provided herein, the imaging is accomplished by confocal microscopy. Confocal fluorescence microscopy involves scanning a focused laser beam across the sample, and imaging the emission from the focal point through an appropriately-sized pinhole. This suppresses the unwanted fluorescence from sections at other depths in the sample. In embodiments, the imaging is accomplished by multi-photon microscopy (e.g., two-photon excited fluorescence or two-photon-pumped microscopy). Unlike conventional single-photon emission, multi-photon microscopy can utilize much longer excitation wavelength up to the red or near-infrared spectral region. This lower energy excitation requirement enables the implementation of semiconductor diode lasers as pump sources to significantly enhance the photostability of materials. Scanning a single focal point across the field of view is likely to be too slow for many sequencing applications. To speed up the image acquisition, an array of multiple focal points can be used. The emission from each of these focal points can be imaged onto a detector, and the time information from the scanning mirrors can be translated into image coordinates. Alternatively, the multiple focal points can be used just for the purpose of confining the fluorescence to a narrow axial section, and the emission can be imaged onto an imaging detector, such as a CCD, EMCCD, or s-CMOS detector. A scientific grade CMOS detector offers an optimal combination of sensitivity, readout speed, and low cost. One configuration used for confocal microscopy is spinning disk confocal microscopy. In 2-photon microscopy, the technique of using multiple focal points simultaneously to parallelize the readout has been called Multifocal Two-Photon Microscopy (MTPM). Several techniques for MTPM are available, with applications typically involving imaging in biological tissue. In embodiments of the methods provided herein, the imaging is accomplished by light sheet fluorescence microscopy (LSFM). In embodiments, detecting includes 3D structured illumination (3DSIM). In 3DSIM, patterned light is used for excitation, and fringes in the Moiré pattern generated by interference of the illumination pattern and the sample, are used to reconstruct the source of light in three dimensions. In order to illuminate the entire field, multiple spatial patterns are used to excite the same physical area, which are then digitally processed to reconstruct the final image. See York, Andrew G., et al. “Instant super-resolution imaging in live cells and embryos via analog image processing.” Nature methods 10.11 (2013): 1122-1126 which is incorporated herein by reference. In embodiments, detecting includes selective planar illumination microscopy, light sheet microscopy, emission manipulation, pinhole confocal microscopy, aperture correlation confocal microscopy, volumetric reconstruction from slices, deconvolution microscopy, or aberration-corrected multifocus microscopy. In embodiments, detecting includes digital holographic microscopy (see for example Manoharan, V. N. Frontiers of Engineering: Reports on Leading-edge Engineering from the 2009 Symposium, 2010, 5-12, which is incorporated herein by reference). In embodiments, detecting includes confocal microscopy, light sheet microscopy, or multi-photon microscopy.

The cell or tissue may be manipulated prior to immobilizing the cell or tissue onto a solid support using known techniques in the art (see, e.g., PCT Publication WO2023076832A1). In embodiments, the method further includes cutting a sample portion from the biological sample (e.g., including cells or tissues) using a punch device such that the punch device contains the sample portion; mounting the punch device containing the sample portion onto a substrate or support as described herein (e.g., inverting the punch device); pushing the sample portion out of the punch device using a piston, so that all or a portion thereof of the sample portion is positioned on a substrate or support as described herein. In embodiments, the method further includes cutting a sample portion from the biological sample using two or more punch devices such that each punch device contains a different the sample portion; mounting each punch device containing the sample portion onto a substrate or support as described herein; pushing the sample portions out of the punch devices using one or more pistons so that the sample portions are positioned onto a substrate or support as described herein.

In embodiments, the cell or tissue is permeabilized and immobilized to a solid support surface. In embodiments, the cell or tissue is attached to a solid support described herein. In embodiments, the tissue is a tissue section. In embodiments, the tissue includes a plurality of cells. In embodiments, the method includes immobilizing 24 tissue sections (10 mm×17 mm sections). In embodiments, the method includes immobilizing 40 tissue sections (10 mm×10 mm sections). In embodiments, the method includes immobilizing 128 tissue sections (4 μm×4 μm sections).

In embodiments, the cell is immobilized to a substrate. The cell may have been cultured on the surface, or the cell may have been initially cultured in suspension and then fixed to the surface. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished as described herein, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.”

In embodiments, the solid support includes a glass substrate. In embodiments, the glass substrate is a borosilicate glass substrate with a composition including SiO₂, Al₂O₃, B₂O₃, Li₂O, Na₂O, K₂O, MgO, CaO, SrO, BaO, ZnO, TiO₂, ZrO₂, P₂O₅, or a combination thereof (see e.g., U.S. Pat. No. 10,974,990). In embodiments, the glass substrate is an alkaline earth boro-aluminosilicate glass substrate. In embodiments, the solid support or substrate described herein includes one or more channels. In embodiments, the solid support or substrate includes a channel bored into solid support or substrate. In embodiments, the solid support or substrate includes a plurality of channels solid support or substrate. In embodiments, the solid support or substrate includes 2, 3, or 4 channels bored into solid support or substrate. In embodiments, the width of the channel is from about 1 to 5 mm, 5 mm to 10 mm, or 10 mm to 15 mm. In embodiments, the channel is a reaction chamber on the solid support or substrate. In embodiments, the cell or tissue is immobilized in a channel bored onto the solid support or substrate.

In embodiments, the cell is exposed to paraformaldehyde (i.e., by contacting the cell with paraformaldehyde). Any suitable permeabilization and fixation technologies can be used for making the cell available for the detection methods provided herein. In embodiments the method includes affixing single cells or tissues to a transparent substrate. Exemplary tissue include those from skin tissue, muscle tissue, bone tissue, organ tissue and the like. In embodiments, the method includes immobilizing the cell in situ to a substrate and permeabilized for delivering probes, enzymes, nucleotides and other components required in the reactions. In embodiments, the cell includes many cells from a tissue section in which the original spatial relationships of the cells are retained. In embodiments, the cell in situ is within a Formalin-Fixed Paraffin-Embedded (FFPE) sample. In embodiments, the cell is subjected to paraffin removal methods, such as methods involving incubation with a hydrocarbon solvent, such as xylene or hexane, followed by two or more washes with decreasing concentrations of an alcohol, such as ethanol. The cell may be rehydrated in a buffer, such as PBS, TBS or MOPs. In embodiments, the FFPE sample is incubated with xylene and washed using ethanol to remove the embedding wax, followed by treatment with Proteinase K to permeabilized the tissue. In embodiments, the cell is fixed with a chemical fixing agent. In embodiments, the chemical fixing agent is formaldehyde or glutaraldehyde. In embodiments, the chemical fixing agent includes both formaldehyde and glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE®, CytoSkelFix™, F-Solv®, FineFIX®, RCL2/KINFix, UMFIX, Glyo-Fixx®, Histochoice®, or PAXgene®. In embodiments, the cell is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM).

In embodiments the cell is lysed to release nucleic acid or other materials from the cells. For example, the cells may be lysed using reagents (e.g., a surfactant such as Triton®-X or SDS, an enzyme such as lysozyme, lysostaphin, zymolase, cellulase, mutanolysin, glycanases, proteases, mannase, proteinase K, etc.) or a physical lysing mechanism a physical condition (e.g., ultrasound, ultraviolet light, mechanical agitation, etc.). The cells may release, for instance, DNA, RNA, mRNA, proteins, or enzymes. The cells may arise from any suitable source. For instance, the cells may be any cells for which nucleic acid from the cells is desired to be studied or sequenced, etc., and may include one, or more than one, cell type. The cells may be for example, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally-occurring sample (e.g., pond water, soil, etc.), or the like. In some cases, the cells may be dissociated from tissue. In embodiments, the method does not include dissociating the cell from the tissue or the cellular microenvironment. In embodiments, the method does not include lysing the cell.

In embodiments, the method further includes subjecting the cell to expansion microscopy methods and techniques. Expansion allows individual targets (e.g., mRNA or RNA transcripts) which are densely packed within a cell, to be resolved spatially in a high-throughput manner. Expansion microscopy techniques are known in the art and can be performed as described in US 2016/0116384 and Chen et al., Science, 347, 543 (2015), each of which are incorporated herein by reference in their entirety.

In embodiments, the method does not include subjecting the cell to expansion microscopy. Typically, expansion microscopy techniques utilize a swellable polymer or hydrogel (e.g., a synthetic matrix-forming material) which can significantly slow diffusion of enzymes and nucleotides. Matrix (e.g., synthetic matrix) forming materials include polyacrylamide, cellulose, alginate, polyamide, cross-linked agarose, cross-linked dextran or cross-linked polyethylene glycol. The matrix forming materials can form a matrix by polymerization and/or crosslinking of the matrix forming materials using methods specific for the matrix forming materials and methods, reagents and conditions known to those of skill in the art. Additionally, expansion microscopy techniques may render the temperature of the cell sample difficult to modulate in a uniform, controlled manner. Modulating temperature provides a useful parameter to optimize amplification and sequencing methods. In embodiments, the method does not include an exogenous matrix.

In an aspect is provided a method of detecting a disorder (e.g., cancer) or a disease-causing mutation or allele in a cell. In embodiments, the cell includes an oncogene (e.g., HER2, BRAF, EGFR, KRAS) and utilizing the methods described herein the oncogene is identified, thereby detecting a disorder when the presence of the oncogene is identified. In embodiments, the sample includes a nucleic acid molecule which includes a disease-causing mutation or allele. In embodiments, the method includes hybridizing an oligonucleotide primer which is correlated with the disease-causing mutation or allele. In embodiments, the method includes ligating a mutation-specific oligonucleotide only when the disease-causing mutation or allele is present in the nucleic acid target. In embodiments, the disease-causing mutation or allele is a base substitution, an insertion mutation, a deletion mutation, a gene amplification, a gene deletion, a gene fusion event, or a gene inversion event.

In embodiments, the mutation or allele is associated with an increased predisposition for one or more diseases, disorders, or other phenotypes. In embodiments, the mutation or allele is associated with a decreased predisposition for one or more diseases, disorders, or other phenotypes. For example, some mutations or alleles are associated with a cancer phenotype, such as decreased growth inhibition, evasion of immune detection, or dedifferentiation. Mutations that can be detected using the method provided herein include for example, mutations to BRAF, EGFR, Her2/ERBB2, and other somatic mutations as exemplified by Greenman et al., Nature (2007) 446:153-158, hereby incorporated by reference in its entirety.

In embodiments, the tissue may further be stained. The sample can be stained using hematoxylin and eosin (H&E) staining techniques, using Papanicolaou staining techniques, Masson's trichrome staining techniques, silver staining techniques, Sudan staining techniques, immunofluorescence (IF) staining techniques (e.g., an immunofluorescence label conjugated to an antibody), and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and/or Giemsa stain.

### Examples

Simultaneous detection of different nucleic acid targets in situ, particularly when there are significant disparities in the expression levels of those different targets, has historically posed several challenges. For example, in cases where some targets are expressed at much higher quantities than others, there is a risk of signal overlap and/or oversaturation when using fluorescent based detection. High-abundance targets can produce strong signals that overshadow or mask the detection of low-abundance targets. This wide dynamic range imbalance makes it difficult to accurately detect and quantify multiple targets within the same sample.

Quantitatively analyzing targets with disparate abundance levels is challenging. High-abundance targets can easily be quantified, but accurately quantifying low-abundance targets in the presence of high-abundance ones requires highly sensitive and specific detection methods that can differentiate between the varying levels of expression. Maintaining high spatial resolution for in situ detection is essential to understand the localization and distribution of targets within the tissue context. However, the co-localization of high and low-abundance targets often complicates this, as the intense signal from abundant targets can obscure the precise localization of less abundant ones. Designing assays that can simultaneously accommodate the detection of targets with vastly different expression levels is technically challenging. This often involves optimizing probe affinity, amplification conditions, and detection thresholds, which can be a complex and iterative process.

Additionally, the simultaneous detection of nucleic acid molecules and proteins in situ has historically faced several challenges due to the inherent complexities of combining molecular techniques that are typically used separately for detecting these different types of biomolecules. For example, nucleic acids and proteins require distinct detection methods. Nucleic acids are often detected using techniques like fluorescence in situ hybridization (FISH) or in situ sequencing approaches (e.g., as described U.S. Pat. Nos. 11,492,662; 11,643,679; 11,434,525; 11,680,288; and/or U.S. Pat. No. 11,753,678), while proteins are commonly identified through immunohistochemistry (IHC), immunofluorescence, or antibody-oligo conjugates (e.g., as described in U.S. Pat. No. 11,643,679 or U.S. Pat. No. 11,753,678). Integrating these methods into a single, coherent protocol that can simultaneously target both molecules without interference is complex.

One means for overcoming the above is the use of protein-specific probes (e.g., antibody-oligonucleotide conjugates). Such probes can be used in conjunction with nucleic acid-based probes, amplified, and detected using similar methods. However, this still does not address problems that arise when there are significant disparities in the levels of those different targets. There exists a clear need for an integrated approach that can simultaneously amplify and detect signals from low-abundance targets without compromising the detection of high-abundance targets, ensuring that signals from high-abundance targets do not saturate the detection system or mask the presence of less abundant molecules.

The invention described herein addresses these challenges. For example, a method described herein provides a novel approach for differential amplification of nucleic acids. This method could be adapted or combined with protein detection techniques, to allow for simultaneous in situ analysis.

### Example 1. Components for Detecting Proteins and Nucleic Acids In Situ Using Circularized Polynucleotide Probes

An approach described herein utilizes at least two different circularizable probes (which, in embodiments, may be referred to herein as padlock probes (PLPs) or single-stranded polynucleotides described herein), at least one of which is modulated with one or more retarding agent(s). The retarding agent may be located within the padlock probe itself (e.g., a modified nucleic acid or hairpin loop), or could be an agent that selectively binds one probe to reduce amplification. The retarding agent effectively slows down the amplification rate of the probe it is associated with.

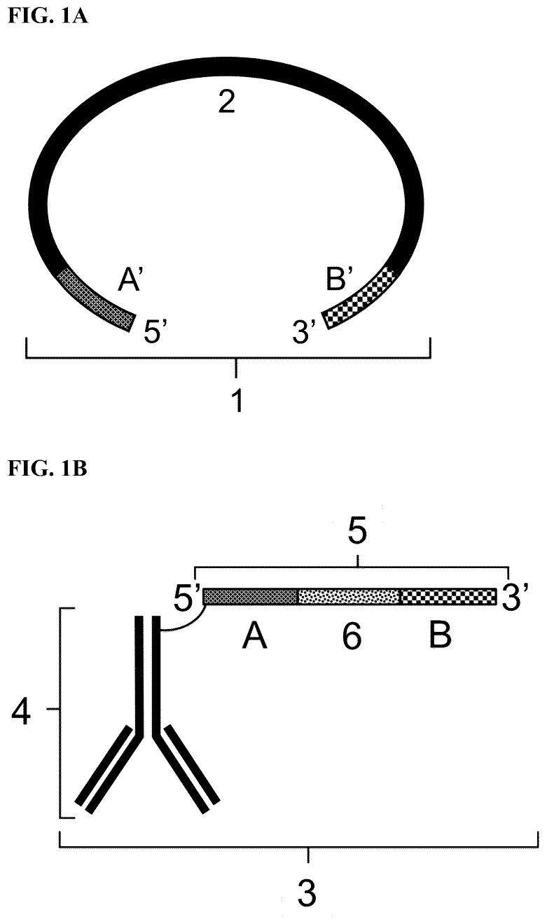

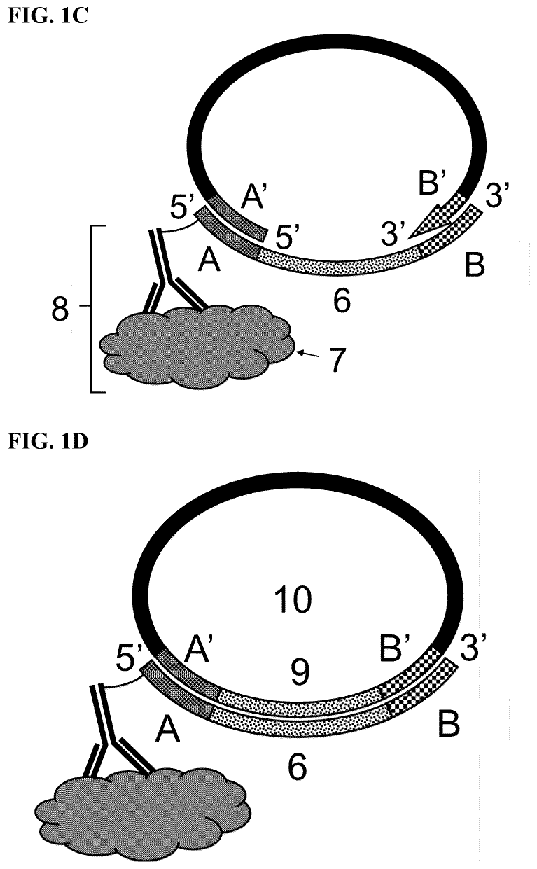

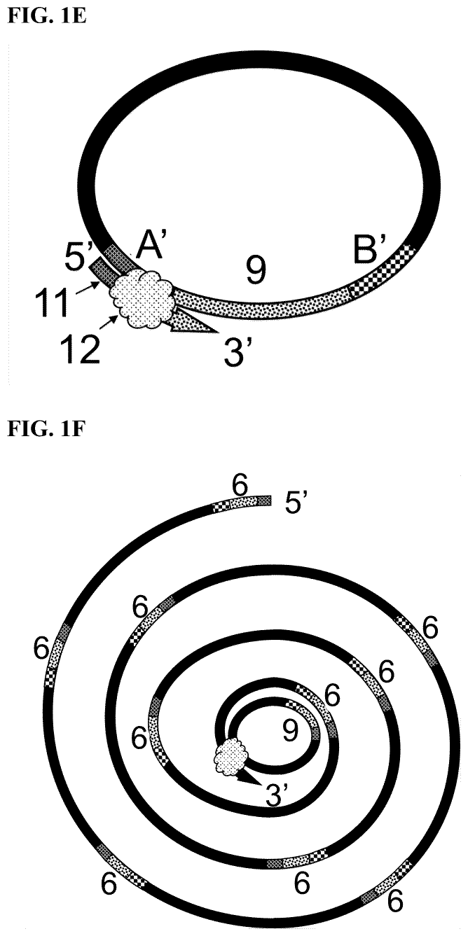

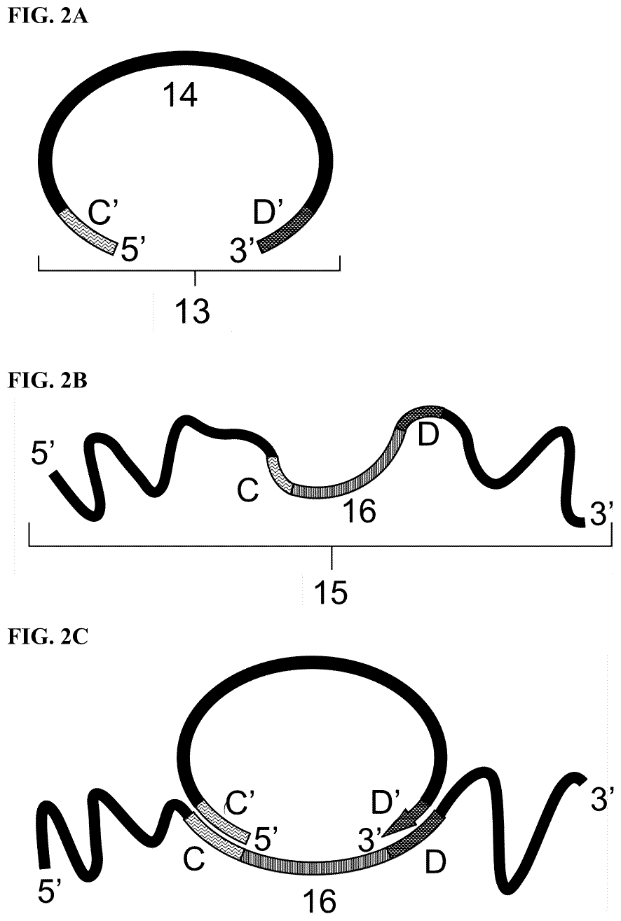

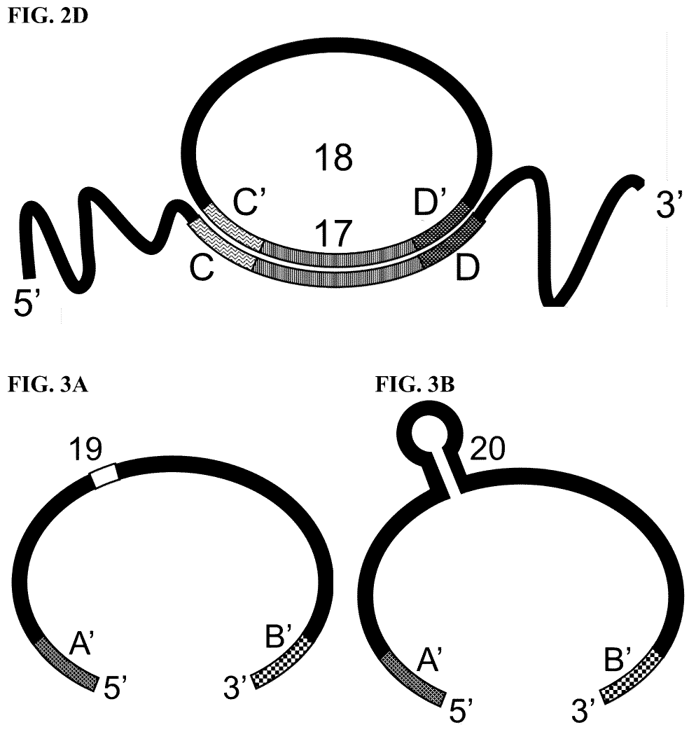

Nonlimiting examples of circularizable probes for in situ detection of nucleic acid or protein target molecules are illustrated in FIGS. 1A and 2A. In both examples, the probe (e.g., the single stranded polynucleotide described herein) includes a linker region flanked by a 5′ (labeled as A′) and 3′ (labeled as B′) arm region. Here, the linker region serves to connect the 5′ and 3′ arms such that circularization of the probe occurs once the ends are either ligated directly to each other or filled (as is shown in FIGS. 1D and 2D), following binding to the target molecule. The linker can also include additional functional sequences, such as a primer binding sequence (e.g., a region of complementarity to an amplification and/or sequencing primer), a barcode (e.g., one or more identifying nucleotides), and/or a probe binding sequence (e.g., a complementary sequence for a fluorescently labelled probe).

Nonlimiting examples of probes for in situ detection of nucleic acid and/or protein target molecules which include a retarding agent are illustrated in FIGS. 3A and 3B. In the example illustrated in FIG. 3A, the retarding agent includes one or more modified nucleotides (19) incorporated into the probe linker region. It is understood that the one or more modified nucleotides may be distributed throughout the probe sequence (e.g., in the A′, B′ and/or linking sequence). The modified nucleotides may be consecutive (i.e., immediately adjacent to each other) or may be present according to a set interval (e.g., 10 native nucleotides, one modified nucleotide, 10 native nucleotides, etc.). Alternatively, in embodiments, the modified nucleotides may be interspersed in no particular order or arrangement (e.g., random location(s)) throughout the probe polynucleotide. In FIG. 3B, the retarding agent is depicted as a hairpin loop (20) which is also incorporated into the linker region. Secondary structures, such as a hairpin or duplex region, serves to stall or retard the polymerase from amplifying the polynucleotides.

Retarding agents modulate amplification rates for associated probes as illustrated in FIG. 4A and FIG. 4B. In each example figure, the number of copies of the target sequences in amplification products generated simultaneously with modified probes (four copies, as seen in FIGS. 4A and 4B) in this lower than those from the unmodified probes (eight copies, as seen in FIGS. 4A and 4B). In these particular examples, the retarding agent is either a modified nucleotide (FIG. 4A), or a hairpin loop (FIG. 4B).

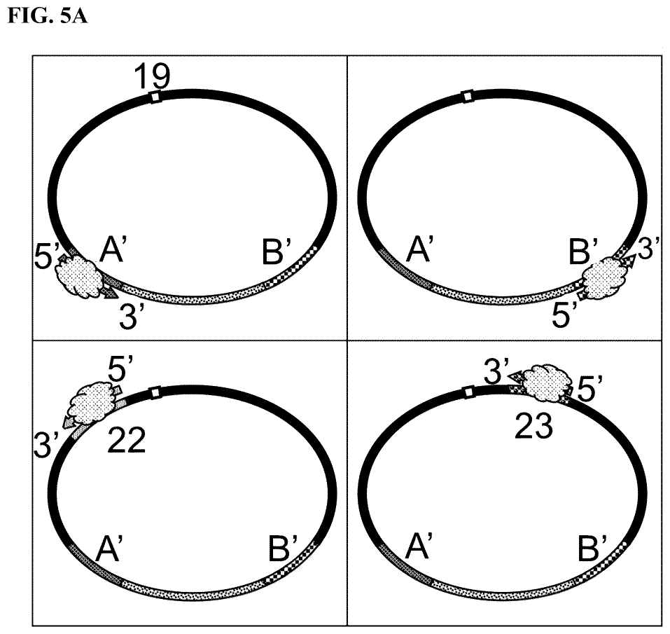

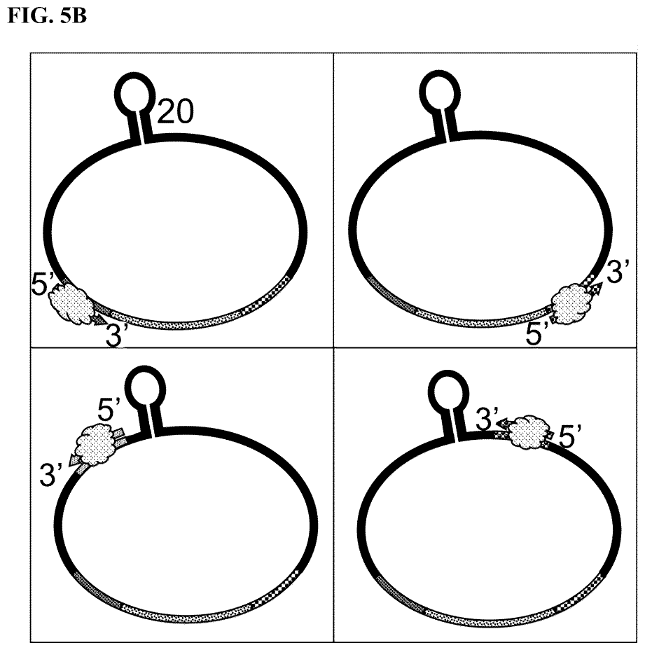

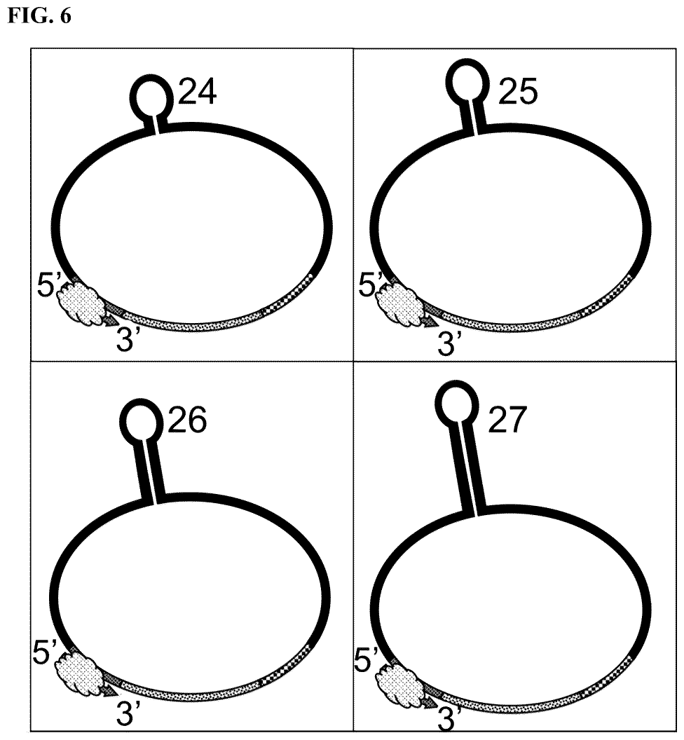

FIGS. 5A and 5B illustrate another embodiment where the placement of the amplification primer binding sequence within the circular polynucleotide relative to the location of the retarding agent can be varied in order to have further control of the amplification rate of the closed circular polynucleotide. FIG. 6 illustrates another embodiment where the length of the double-stranded region in the circular polynucleotide is varied as yet another means of fine tuning the amplification rate.

Proteins are typically found in higher abundance than RNA molecules within a cell. FIG. 7 illustrates a nonlimiting example of how the combination of the components described within, facilitate the simultaneous detection of proteins and nucleic acids, within the same sample, by differentially amplifying target sequences at controlled rates, enabling distinct identification and quantification. Reducing the amplification output from a protein probe relative to a nucleic acid probe enables simultaneous amplification and quantification of proteins and transcripts in the same cell or tissue.

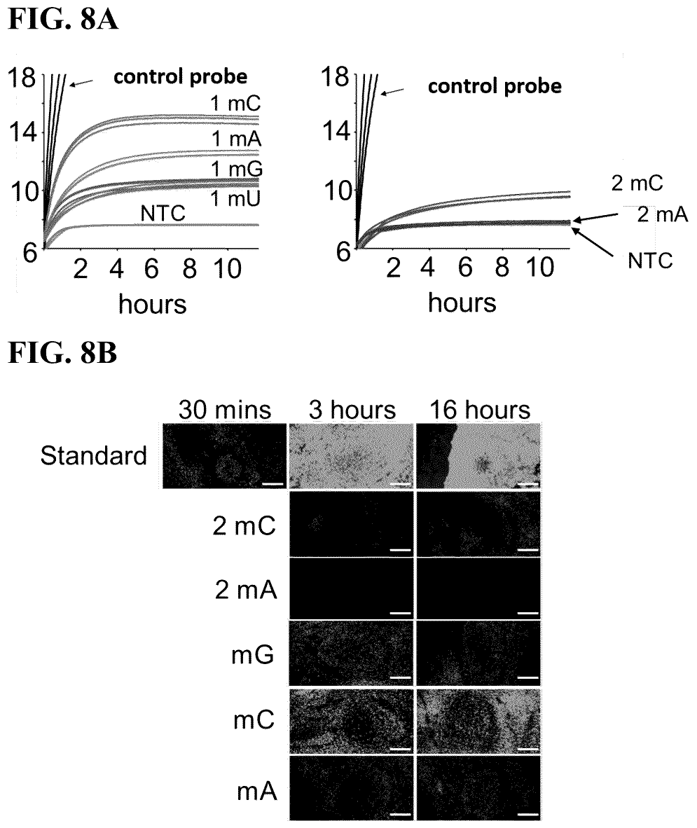

FIGS. 8A-8B provide experimental evidence of reduced amplification. We designed probes containing one or two consecutive 2′OMe modified bases and subjected them to intramolecular ligation in vitro to generate single stranded DNA circles. The circular probes labeled 1 mC (or mC) corresponds to a probe including a 2′OMe modified cytosine; 1 mA (or mA) corresponds to a probe inclusive of a 2′OMe modified adenine; 1 mG (or mG) corresponds to a probe inclusive of a 2′OMe modified guanine; 1 mU (or mU) corresponds to a probe inclusive of a 2′OMe modified uracil; 2 mC corresponds to a probe inclusive of two consecutive 2′OMe modified cytosines; 2 mA corresponds to a probe inclusive of two consecutive 2′OMe modified adenines; and NTC is the non-template control probe used for the assay. The efficiency of rolling circle amplification on each padlock probe design was assayed by continuous monitoring of phi29 amplicon products using SYTO™ 9 fluorescent dye over a 12 hour period compared to a control probe consisting of natural bases. FIG. 8A shows the detected fluorescence over time, and shows presence of a retarding agent (e.g., 1 mC, 1 mA, 1 mG, and 1 mU) slows amplification relative to the control probe. Additionally, probes including two consecutive retarding agents (e.g., 2 mA) significantly impacts amplification, measurably indistinct from the NTC. The 2′OMe probe designs were then evaluated using fluorescence in situ hybridization assays to evaluate amplicon signal intensity and antigen labeling efficiency following 3 or 16 hours of in situ RCA (FIG. 8B). The white scale bar of each image corresponds to 200 μm. The relative abundance of the desired target may factor into the choice of the identity and quantity of the retarding agent.

### Example 2. Simultaneous Detection of KRAS and TP53 in Colorectal Cancer

In certain types of cancer, such as colorectal cancer, there is a need to simultaneously detect an oncogene like KRAS and a tumor suppressor gene like TP53. These genes play crucial roles in cell growth and apoptosis, respectively, and their mutations are often key indicators of cancer progression and patient prognosis. However, the challenge arises due to the difference in their expression levels. Oncogenes like KRAS can be highly expressed in cancer cells, leading to an abundance of KRAS mRNA transcripts in the tumor tissue. This high expression level results in a strong signal when detected using in situ methods of detection. On the other hand, tumor suppressor genes like TP53 may have lower expression levels, particularly in cancer cells where these genes might be mutated or partially silenced. As a result, the TP53 mRNA transcripts are present in much lower quantities compared to KRAS, leading to weaker detection signals. When attempting to simultaneously detect these two genes in a single tissue sample, the strong signal from the abundant KRAS transcripts can overshadow the weaker signal from the TP53 transcripts. This disparity makes it challenging to accurately assess the presence and quantity of TP53, which is crucial for understanding the tumor's behavior and potential response to treatment. Traditional in situ hybridization techniques might not have the sensitivity or specificity required to effectively differentiate between the high signals from KRAS and the low signals from TP53. This can lead to misinterpretation or underestimation of TP53 expression levels.

The methods described herein offer an innovative approach to simultaneously detect genes like KRAS and TP53 in situ, particularly in contexts where KRAS may be overexpressed relative to TP53, such as in colorectal cancer. This approach involves the use of a retarding agent to modulate the amplification rate of the probe targeting KRAS, ensuring that the KRAS signal does not overwhelm the TP53 signal.

In this example, separate probes are designed for KRAS and TP53. The KRAS probe is tailored to hybridize specifically to a commonly mutated or overexpressed sequence in KRAS and includes one or more retarding agents. The TP53 probe is designed to target a sequence indicative of its tumor suppressor function, but does not necessarily include retarding agents. The retarding agent may be a modified nucleotide, as illustrated in FIG. 3A, or a hairpin structure as illustrated in FIG. 3B, that slows down the amplification process, reducing the intensity of the KRAS signal (e.g., when detecting via fluorescence) to a signal level more comparable with TP53. The colorectal cancer tissue sample is treated with both KRAS and TP53 probes. The probes hybridize in situ to their respective gene targets. Rolling Circle Amplification (RCA) is initiated for both sets of probes concurrently (see, e.g., FIGS. 2C and 2D). The retarding agent in the KRAS probe modulates its amplification rate, allowing the TP53 probe to be amplified at a relatively faster rate. This differential amplification prevents the KRAS signal from dominating the TP53 signal upon detection (e.g., by sequencing, see, e.g., FIG. 2B).

This method allows for a more nuanced understanding of the cancer's genetic profile, which is crucial for prognosis and determining the most effective treatment strategy. The ability to simultaneously and accurately quantify the expression of both KRAS and TP53 in situ is particularly valuable for personalized medicine approaches in oncology.

### Example 3. Detection of EGFR Mutations and PDL1 Protein Expression in Non-Small Cell Lung Cancer (NSCLC)

In NSCLC, two critical biomarkers often assessed for diagnostic and therapeutic purposes are EGFR (Epidermal Growth Factor Receptor) mutations in the tumor's DNA and the expression of the PDL1 (Programmed Death-Ligand 1) protein. In some NSCLC tumors, there can be an overexpression of the PDL1 protein, often as a mechanism by which the cancer evades the immune system. This overexpression results in a high abundance of PDL1 proteins within the tumor microenvironment. Contrarily, specific mutations in the EGFR gene, which are critical for targeted therapies, may be present in lower quantities. These mutations are often focal and may not be uniformly distributed across all tumor cells. When attempting to detect both PDL1 protein and EGFR mutations simultaneously in situ, the abundant PDL1 protein expression can overwhelm the emission signal from the less abundant EGFR mutations. This imbalance makes it challenging to accurately assess the presence and quantity of EGFR mutations. The techniques used for protein detection, such as immunohistochemistry (IHC), may not be sensitive enough to detect low-abundance genetic mutations. Conversely, methods used for detecting EGFR mutations, such as in situ hybridization, may not effectively quantify high levels of protein expression. There is also a potential for interference between the detection reagents and protocols used for identifying proteins and nucleic acids, especially when one target is much more abundant than the other.

The methods described herein can be utilized to simultaneously detect the EGFR mutations and PDL1 protein expression in situ in non-small cell lung cancer (NSCLC), even when PDL1 is in higher abundance. This can be achieved by incorporating a retarding agent to modulate the amplification rate of the probe used for detecting the PDL1 protein, ensuring the PDL1 signal does not overwhelm the EGFR signal when detecting fluorescent events.

In this example, separate probes are designed for detecting EGFR mutations, with sequences complementary to the mutation sites. Concurrently, antibody-oligonucleotide conjugates, an example of which is illustrated in FIG. 1B, targeting the PDL1 protein are developed, where the antibody specifically binds to the PDL1 protein and the attached oligonucleotide serves as a hybridization sequence for a second probe, a nonlimiting example of which is illustrated in FIG. 7, upper left. To address the higher abundance of PDL1, a retarding agent is integrated into the probe targeting the oligo of the antibody-oligo conjugate targeting PDL1. The retarding agent, such as a modified nucleotide or hairpin structure, is designed to slow down the amplification process of this probe, thereby reducing the intensity of the PDL1 signal. The lung cancer tissue sample is treated with both the EGFR-specific probes and the PDL1-targeted antibody-oligonucleotide conjugates (a nonlimiting example of which is illustrated in FIG. 7 (top). Upon binding to their respective targets, the rolling circle amplification (RCA) process is initiated (e.g., see FIGS. 1C-IF). Due to the presence of the retarding agent, the amplification of the extension product associated with PDL1 is slowed, allowing the extension product associated with the EGFR mutation to be amplified at a relatively faster rate, resulting in an amplicon with a lower number of copies of the PDL1 detection sequence (FIG. 7, bottom left) relative to that of the EGFR mutation detection sequence (FIG. 7, bottom right). This controlled amplification prevents the PDL1 signal, despite its higher abundance, from overwhelming the EGFR mutation signal upon fluorescent detection (e.g., by sequencing the extension products). This method thus allows for the accurate quantification of these biomarkers, which is crucial for treatment decision-making, such as determining the suitability of EGFR-targeted therapies and immunotherapies.

### Example 4. Modifications to Oligonucleotide Probe and RCA

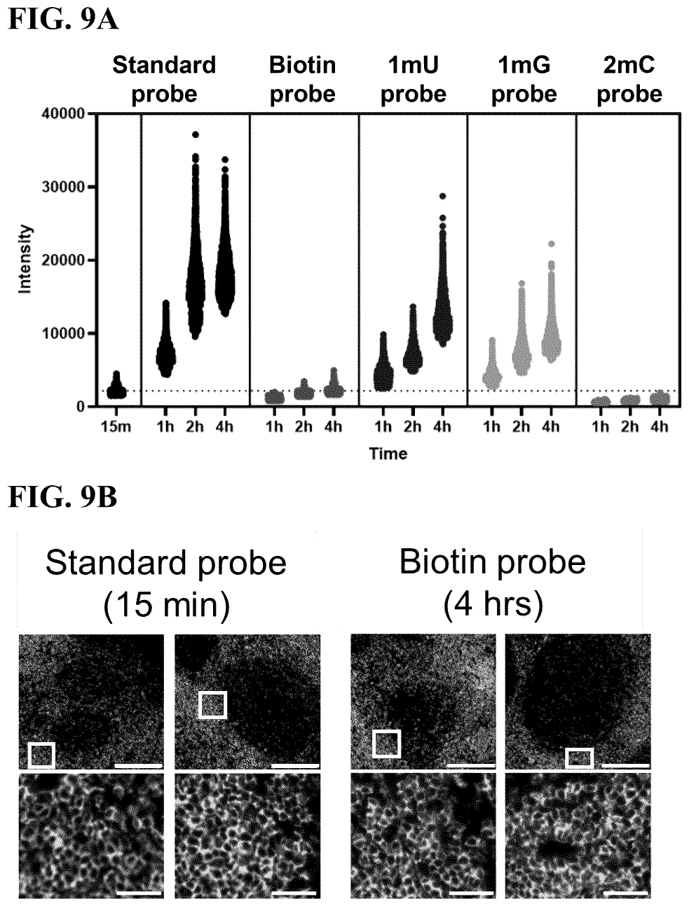

Here we explore the impact of different modifications to the circular polynucleotide and the downstream impacts of amplification. Shown in FIG. 9A, is the fluorescent in situ hybridization (FISH) and quantification of amplification products for various time points when detecting CD3e proteins in tonsil. Antibody-oligo (Ab-O) conjugates specific for CD3a proteins were incubated using standard staining conditions. Circularizable probes targeting the oligonucleotide sequences of the Ab-O were incubated and amplified. An unmodified circular polynucleotide (referred to as the standard probe) amplified for 15 minutes and the detection products was quantified. Amplification products for a biotin probe, 1 mU probe, 1 mG probe and 2 mC probe were detected at 1 hour, 2 hour, and 4 hour time intervals. These modifications are 5′ of the primer binding site. This experiment was performed in duplicate for each timepoint on non-adjacent sections. FIG. 9A shows that 4 hours of rolling circle amplification (RCA) with a biotinylated probe produced similar quantities of amplification product at the 15 minutes RCA with unmodified probe for CD3e. Additionally, we confirm that biotinylated padlock effectively reduces the rate of RCA without hindering cell detection or cell morphology; see FIG. 9B.

By leveraging this differential amplification strategy, it is possible to balance the detection of targets with disparate initial abundance levels. For example, a high-abundance target could be detected using a standard unmodified probe with a short RCA time, ensuring robust signal generation, followed by termination with a ddNTP to halt extension. Next, a low-abundance target could be detected with a biotinylated probe and a longer RCA duration, preventing the high-abundance target signal from overshadowing the low-abundance target's signal. Alternatively, employing a biotinylated probe for a high-abundance target and a standard unmodified probe for a low-abundance target allows for effective signal management. The slower amplification rate of the biotinylated probe prevents signal oversaturation from the high-abundance target, ensuring its signal remains within a manageable range. Concurrently, the faster amplification of the unmodified probe enhances the detectability of the low-abundance target. This dual-probe strategy improves the dynamic range of detection, enabling accurate and simultaneous quantification of both high and low-abundance nucleic acid targets within the same sample, thus overcoming significant challenges associated with traditional fluorescent detection techniques.

The addition of streptavidin during amplification effectively halted (i.e., arrests) amplification. This is important because it suggests that protein targets can be amplified and then blocked with streptavidin, allowing RNA RCA to continue for the required amplification time. As illustrated in FIGS. 9A-9B, the biotinylation of circular polynucleotide probes was shown to significantly slow RCA, producing comparable amplification products at 4 hours as the unmodified probes did at 15 minutes. Moreover, our findings reveal that the addition of streptavidin during RCA can further decrease the amplification rate of biotinylated padlocks and even completely inhibit RCA if streptavidin is introduced before RCA initiation. This discovery enables a novel approach to multiplex target detection.

For example, a method of arresting amplification may be useful during the simultaneous detection of RNA and protein targets in situ. In a sample where both a high-abundance RNA target and a protein target need to be detected, we may utilize a biotinylated probe for the protein target and a standard unmodified probe for the RNA target. During the initial stages of RCA, the biotinylated probe would undergo a slower amplification, preventing oversaturation. After a predetermined amplification period (e.g., 15 minutes) streptavidin is added to the system to arrest further amplification of the biotinylated probe, effectively “freezing” the amount of amplification products and thus the detected signal for the protein target. This halting mechanism ensures that the protein target signal does not overshadow the RNA target detection. Following the arrest of the protein target amplification, the RCA for the RNA target, facilitated by the unmodified probe, would continue unhindered for the required duration to achieve sufficient amplification. This method allows the RNA target to reach detectable levels without interference from the protein target's signal. By timing the addition of streptavidin, this approach ensures that both high-abundance and low-abundance targets are accurately and simultaneously detected, thereby enhancing the dynamic range and reliability of in situ hybridization assays.

### Example 5. Serial Revealing Cell Paints

The detection and analysis of multiple biomolecules within the same cell or tissue section is crucial for understanding the phenotypic and functional architecture of healthy and diseased states. Traditional single-plex techniques, such as enzyme-linked immunosorbent assays (ELISA), are limited in their ability to provide comprehensive insights due to their focus on single analytes. In contrast, multiplexed detection methods offer the potential to simultaneously analyze multiple biomarkers, thereby providing a more holistic view of cellular and tissue states. However, existing multiplexed antibody-based techniques, including those employing fluorophores, metal markers, and DNA barcodes, face significant challenges. These challenges include the need for meticulous antibody validation, issues with spectral overlap, and the complex and time-consuming nature of sequential staining and bleaching protocols.

Fluorescent multiplexing techniques, such as multiplex immunofluorescence and tissue-based circular immunofluorescence, rely on labeling biomolecules with distinct fluorophores. While these methods can provide sensitive and specific detection, they are constrained by the limited spectral range available for fluorescence detection. Spectral overlap occurs when fluorophores emit light at similar wavelengths, making it difficult to distinguish between different targets. This overlap necessitates the use of dyes with minimal emission overlap, often restricting the number of biomarkers that can be simultaneously detected to four or five. Additionally, methods for removing or inactivating fluorophores after each round of staining, such as enzymatic digestion or chemical bleaching, can damage the sample and prolong the imaging process. As a result, detecting a large number of targets can become impractically lengthy and complex.

To address these limitations, advanced techniques like DNA barcoding have been developed, enabling higher multiplexing capabilities by avoiding spectral limitations. However, these methods typically require complex probe design and hybridization protocols, which can be cumbersome and expensive. Additionally, despite these advances, the sole reliance on antibodies remains a hurdle, as it requires rigorous validation to ensure accuracy and reproducibility. Consequently, there is a pressing need for innovative approaches that can overcome these challenges, enabling efficient and accurate multiplex detection of biomolecules in situ, without the drawbacks associated with current technologies.

The present disclosure addresses the limitations of existing multiplexed biomolecule detection methods by introducing an advanced cell painting technique, capable of being used existing spatial biology platforms (e.g., the G4X™ Platform or ImageXpress® Confocal HT.ai system). This innovative approach combines the principles of traditional cell painting with differential amplification of polynucleotides and sequential staining cycles. By integrating these elements, the invention enables the detection of a significantly larger number of cellular structures and biomarkers within the same sample, overcoming the spectral overlap and optical cross-talk issues inherent in conventional fluorescence-based methods.

Described herein are methods for differentially amplifying polynucleotides, e.g., a first circular polynucleotide including a first sequence as described herein and a second circular polynucleotide including a second sequence as described herein, wherein the first circular polynucleotide is hybridized to a nucleic acid molecule covalently attached to a specific binding agent, wherein the specific binding agent is a cell paint targeting molecule as described herein. The first circular polynucleotide also includes a retarding agent that slows the amplification rate of the first circular polynucleotide, thereby differentially amplifying the first sequence of the first circular polynucleotide compared to the second sequence of the second circular polynucleotide.

The mushroom toxin phalloidin is a small bicyclic peptide consisting of seven amino acids with a molecular weight of 789. Phalloidin binds to both large and small filamentous actin (F-actin) with high affinity, and compared to actin-specific antibodies, the non-specific binding of phalloidin is negligible, thus providing minimal background and high contrast during cellular imaging. Phalloidin-dye conjugates have been described previously, for example Capani et al Journal of Histochemistry & Cytochemistry. 2001; 49(11):1351-1361, and including a cleavable site in the linker to the fluorophore enables the conjugate to be used in the method described herein. For example, the probe may have the structure:

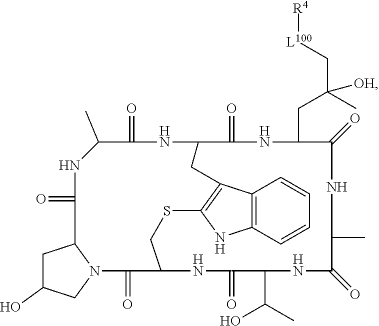

where L¹⁰⁰ is the cleavable linker and R⁴ is a fluorophore moiety.

The method also incorporates automated imaging and image analysis software, enhancing the efficiency and reproducibility of the staining and imaging process. This automation reduces manual intervention, minimizes potential errors, and facilitates large-scale studies. The resulting high-dimensional data can be integrated and analyzed to provide comprehensive profiles of cellular phenotypes, enabling detailed studies of cellular behavior, disease mechanisms, and treatment responses.

The phenotypic profile of a cell reveals the biological state of a cell. More specifically, the phenotypic profile can be used to interrogate biological perturbations because the cellular morphology is influenced by factors such as metabolism, genetic and epigenetic state of the cell, and environmental cues. In addition, it can be used to characterize healthy cells from diseased cells. Because a phenotypic profile is an aggregation of a large number of measurements, it is sensitive to deviations or changes to those features extracted using cellular paints. To create a profile of the cells, all of the features from the different organelles that are imaged and analyzed using commercially available cell imaging software (e.g., CellProfiler™) In morphological profiling, measured features include staining intensities, textural patterns, size, and shape of the labeled cellular structures, as well as correlations between stains across channels, and adjacency relationships between cells and among intracellular structures.

Existing cell paints, described in Table 1, are employed to target specific biomolecules. In current cell painting approaches, fluorescent dyes are conjugated to targeting molecules through covalent bonding, ensuring specific and stable labeling of cellular structures. The attachment process typically involves the use of chemical linkers that form a stable covalent bond between the dye and the targeting molecule. For example, phalloidin, which binds specifically to actin filaments, is covalently linked to a fluorescent dye like Alexa Fluor® 488 using a reactive group on the dye that reacts with a functional group on phalloidin. Similarly, wheat germ agglutinin (WGA), which targets the plasma membrane, is conjugated to a fluorescent dye through a linker that attaches to its glycoprotein-binding sites. This covalent linkage ensures that the dye remains firmly attached to the targeting molecule during the staining, imaging, and any subsequent washing steps, providing consistent and reliable fluorescence labeling of the intended cellular structure.

TABLE 1
Commercially available cell paints

| Targeting Molecule | Fluorescent Dye | Cell Structure Targeted |
|---|---|---|
| Phalloidin | Various (e.g., Alexa Fluor ® 488, | Actin filaments |
| | Alexa Fluor ® 568) | |
| Wheat Germ | Various (e.g., Alexa Fluor ® 488, | Plasma membrane |
| Agglutinin (WGA) | Alexa Fluor ® 594) | |
| MitoTracker ® | Various (e.g., MitoTracker ® Red | Mitochondria |
| | CMXRos, MitoTracker ® Green | |
| | FM) | |
| ER-Tracker ™ | Various (e.g., ER-Tracker ™ Red, | Endoplasmic reticulum |
| | ER-Tracker ™ Green) | |
| Concanavalin A | Various (e.g., Alexa Fluor ® 350) | Endoplasmic reticulum |
| Golgi-Tracker ™ | Various (e.g., BODIPY ® FL C5- | Golgi apparatus |
| | Ceramide) | |
| LysoTracker ® | Various (e.g., LysoTracker ® | Lysosomes |
| | Green DND-26, LysoTracker ® | |
| | Red DND-99) | |
| | CytoFix ™ Red | |
| Annexin V | Various (e.g., Annexin V Alexa | Phosphatidylserine (apoptosis |
| | Fluor ® 488, Annexin V FITC) | marker) |
| Concanavalin A | Various (e.g., Alexa Fluor ® 488, | Cell surface carbohydrates |
| (ConA) | Alexa Fluor ® 594) | |
| Transferrin | Various (e.g., Alexa Fluor ® 488, | Transferrin receptors |
| | Alexa Fluor ® 568) | |
| Lectins (e.g., PNA, | Various (e.g., Alexa Fluor ® 488, | Specific carbohydrate |
| UEA-1) | Alexa Fluor ® 594) | structures |

The method may be useful in detecting biomolecules such as proteins and nucleic acid molecules, organelle structures such as the Golgi Apparatus, and also the cytoskeleton. The cytoskeleton is a network of different protein fibers (e.g., actin and myosin) that maintains the shape and position of the organelles within a cell. The cytoplasm, a fluid which can be rather gel-like, surrounds the nucleus, is considered an organelle.

Additional organelles detectable using the methods and compositions described herein include the Endoplasmic Reticulum (ER), which is a network of membranes that forms channels that cris-crosses the cytoplasm utilizing its tubular and vesicular structures to manufacture various molecules. The ER includes small granular structures called ribosomes useful for the synthesis of proteins. Smooth ER makes fat compounds and deactivates certain chemicals like alcohol or detected undesirable chemicals such as pesticides. Rough ER makes and modifies proteins and stores them until notified by the cell communication system to send them to organelles that require the substances. Typically, all healthy cells in humans, except erythrocytes (red blood cells) and spermatozoa, are equipped with endoplasmic reticulum. The Golgi apparatus (also referred to as a Golgi complex) consists of one or more Golgi bodies which are located close to the nucleus and consist of flattened membranes stacked atop one another like a stack of coins. The Golgi apparatus prepares proteins and lipid (fat) molecules for use in other places inside and outside the cell. Lysosomes are membrane-enclosed organelles that have an acidic interior (pH ˜4.8) and can vary in size from 0.1 to 1.2 μm. Lysosomes house various hydrolytic enzymes responsible for digesting biopolymers such as proteins, peptides, nucleic acids, carbohydrates and lipids. Ribosomes are tiny spherical organelles distributed around the cell in large numbers to synthesize cell proteins. They also create amino acid chains for protein manufacture. Ribosomes are created within the nucleus at the level of the nucleolus and then released into the cytoplasm.

### Example 6. Imaging a Multiplex Tonsil Tissue Sample

To image and analyze a multiplex tonsil tissue sample using a combination of intrinsic (e.g., Hoescht 33342) and non-intrinsic ([targeting molecule]-[cleavable linker (CL)]-[fluorophore]) cell paints as the specific binding agents, cleavable linkers for sequential staining and imaging cycles may be employed. By spatially separating the dyes, we minimize optical cross-talk and maximize detection clarity. To begin, the fixed and prepared tonsil tissue sample is subjected to an initial round of staining using a set of cell paints and immunostains designed to target specific cellular components. The first set includes:
Endoplasmic Reticulum: Concanavalin A (ConA)-CL-Alexa Fluor® 532 (emission: 532 nm)
Golgi Apparatus: Wheat germ agglutinin (WGA)-CL-Alexa Fluor® 594 (emission: 594 nm)
F-Actin: Phalloidin-CL-Alexa Fluor® 647 (emission: 647 nm)
Lysosomes: LysoTracker-CL-Alexa Fluor® 680 (emission: 680 nm)

Once the tissue is stained, it is imaged to capture the fluorescence signals from each dye. Following the initial imaging, the tissue sample undergoes treatment with specific cleavage reagents designed to remove the fluorescent dyes linked through cleavable linkers. The sample is then thoroughly washed to ensure complete removal of the cleaved dyes, preparing it for the next cycle of staining. In the second cycle, the tissue is stained with a new set of cell paints targeting additional structures, each conjugated with non-overlapping dyes to avoid optical cross-talk. This second set includes:
Nucleus: Hoechst 33342 (intrinsic, excitation/emission: 387/447 nm)
Nucleoli: SYTO 14 green fluorescent nucleic acid stain (intrinsic, emission: 531/593 nm)
Mitochondria: MitoTracker™ Deep Red (intrinsic, emission: 628/692 nm)
Transferrin Receptors: Transferrin-CL-Alexa Fluor 532 (emission: 532 nm)
Nuclear Envelope: Anti-Lamin A/C-CL-Alexa Fluor 594 (emission: 594 nm)
Cell Surface Receptors: Anti-CD3-CL-Alexa Fluor 422 (emission: 422 nm)

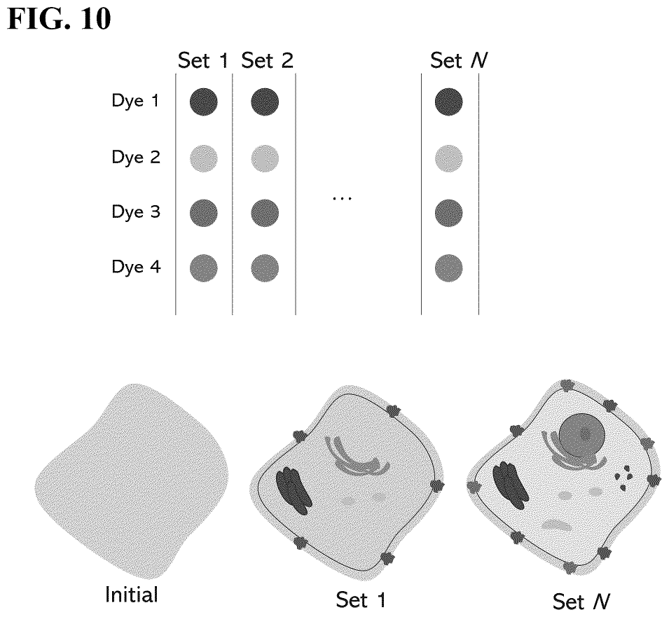

The tissue is then imaged again. After imaging, the dyes are cleaved, and the tissue is prepared for additional cycles, or detection modes, if necessary. This process of staining, imaging, and cleavage is repeated for subsequent cycles, each time introducing new cell paints to target different cellular components as illustrated in FIG. 10. Note, intrinsic stains such as Hoechst 33342 and SYTO 14, should be included in the final set so as not to interfere with detection in intervening staining cycles.

Each cycle ensures that only non-overlapping dyes are used to maintain clear separation of signals. For example, following one or more cycles using the cleavable conjugates described supra one can use traditional (i.e., non-cleavable) staining agents, such as primary antibodies (e.g., beta tubulin monoclonal antibody (ThermoFisher Scientific, 32-2600), anti-clathrin heavy chain antibody (abeam, ab21679), and anti-caveolin-1 antibody (abeam, ab2910) coupled with secondary antibody-oligonucleotide conjugates. For example, protocols for traditional immunostaining may be found Civitci, F. et al. Protoc. Exch. doi.org/10.21203/rs.3.pex-1069/vl (2020).

In addition, the aforementioned cell paints may be covalently attached to a nucleic acid molecule described herein. In embodiments, a circular polynucleotide described herein (e.g., the first circular polynucleotide) may be hybridized to a nucleic acid molecule covalently attached to the cell paint targeting molecule. The first circular polynucleotide also includes a retarding agent that slows the amplification rate of the first circular polynucleotide, thereby differentially amplifying the first sequence of the first circular polynucleotide compared to a second sequence of a second circular polynucleotide. In embodiments, detecting the target molecule (e.g., the first sequence and/or the second sequence as described herein) includes sequencing the amplification product.

After all cycles are completed, the imaging data from each cycle are integrated using commercially available image analysis software. This software aligns the images from different cycles to create a comprehensive map of the cellular structures and biomarkers within the tonsil tissue. The data are then analyzed to quantify the expression and spatial distribution of the targeted components. By sequentially applying cell paints and utilizing cleavable linkers, this method allows for the imaging of a tonsil tissue sample, providing detailed and comprehensive visualization of various cellular components without the limitations of spectral overlap. The high-content imaging system captures high-resolution images, and the integrated data analysis offers insights into the cellular architecture and biomarker distribution within the tissue, facilitating a deeper understanding of tonsil tissue structure and function.

To facilitate the visualization of organelle and related target data commercially available software (e.g., TissueMaker®, TissueFAXS™, THUNDER™) can allow users to dynamically generate a visual interpretation of data. For example, a typical software may present a user interface with a three-dimensional representation of the cell and/or tissue. For example, the method may further include stitching. Stitching combines multiple field of view (FOV) into a single image. Stitching can be performed using a variety of techniques. For example, one approach is, for each row of FOV that together will form the combined image of the sample and each FOV within the row, determine a horizontal shift for each FOV. Once the horizontal shifting is calculated, a vertical shift is calculated for each row of FOV. The horizontal and vertical shifts can be calculated based on cross-correlation, e.g., phase correlation. With the horizontal and vertical shift for each FOV, a single combined image can be generated, and target biomolecule coordinates can be transferred to the combined image based on the horizontal and vertical shift. For the reconstruction of 3D tissues, several computational methods such as PASTE, PASTE2, SLAT, and SPACEL can be utilized. These methods and algorithms typically involve aligning detected targets between different slices and performing coordinate transformation and rotation of different slices to achieve a 3D structure composed of multiple slices. Thus, the use of cell paints as specific binding agents enable targeting organelles and/or cellular components of interest, while the sequencing the amplification product resulting the amplification and hybridization of the polynucleotide probe to the oligonucleotide covalently attached to the cell paint enables detecting the target molecule.

What is claimed is:

1. A method for differentially amplifying polynucleotides in or on a cell or tissue, said method comprising:
(i) contacting a cell or tissue with a plurality of polymerases and deoxynucleotide triphosphates (dNTPs) and
(ii) amplifying a first circular polynucleotide comprising a first sequence to generate an amplification product comprising a first number of copies of the first sequence and amplifying a second circular polynucleotide comprising a second sequence to generate an amplification product comprising a second number of copies of the second sequence; wherein said first number is detectably less than said second number, wherein
the first circular polynucleotide is hybridized to a first nucleic acid molecule covalently attached to a protein-specific binding agent, wherein said first circular polynucleotide comprises a retarding agent; and
the second circular polynucleotide is hybridized to a second nucleic acid molecule.

2. The method of claim 1, wherein the retarding agent is a modified nucleotide, hairpin oligonucleotide, aptamer, or a blocking oligonucleotide bound to the first circular polynucleotide.

3. The method of claim 2, wherein the modified nucleotide is a 2′-O-methyl ribonucleic acid (2′-OMeRNA) nucleotide, biotin-nucleotide, 2′-fluoro ribonucleic acid (2′-F RNA) nucleotide, locked nucleic acid (LNA) nucleotide, or phosphorothioate (PS) nucleotide.

4. The method of claim 2, wherein the modified nucleotide is 5-chloro-2′-deoxyuridine triphosphate, 7-deaza-2′-deoxyadenosine triphosphate, 5-fluoro-2′-deoxycytidine triphosphate, 7-deaza-2′-deoxyguanosine triphosphate, 7-Deaza-7-nitro-dATP, 7-deaza-7-nitro-dGTP, 5-hydroxy-dCTP, 5-hydroxy-dUTP, 5-ethynyl-deoxyuridine triphosphate, or 5′-(α-P-borano)deoxynucleosidetriphosphate.

5. The method of claim 2, wherein the modified nucleotide comprises a bioconjugate reactive moiety.

6. The method of claim 1, wherein the retarding agent is a hairpin oligonucleotide.

7. The method of claim 1, wherein the retarding agent is a blocking oligonucleotide bound to the first circular polynucleotide.

8. The method of claim 1, wherein the protein-specific binding agent is an antibody, single domain antibody, single-chain Fv fragment (scFv), antibody fragment-antigen binding (Fab), affimer, or an aptamer.

9. The method of claim 1, wherein prior to step (i), the method comprises forming said first circular polynucleotide by hybridizing a first end and a second end of a single-stranded polynucleotide to the first nucleic acid molecule and ligating the first end and second end together to form the first circular polynucleotide.

10. The method of claim 1, wherein prior to step (i), the method comprises forming said second circular polynucleotide by hybridizing a first end and a second end of a single-stranded polynucleotide to the second nucleic acid molecule and ligating the first end and second end together to form the second circular polynucleotide.

11. The method of claim 1, wherein prior to step (i), the method comprises forming said second circular polynucleotide by hybridizing a first end and a second end of a single-stranded polynucleotide to the second nucleic acid molecule, extending the second end along the second nucleic acid molecule, and ligating the first end and the extended second end together to form the second circular polynucleotide.

12. The method of claim 1, wherein the first circular polynucleotide and the second circular polynucleotide are each about 50 to about 500 nucleotides.

13. The method of claim 1, wherein the second number is about 1%, about 5%, about 10%, about 15%, about 20%, about 25%, about 30%, about 40%, about 50%, about 75% more than said first number.

14. The method of claim 1, wherein the second number is about 2-fold, at least about 1.5-fold, at least about 2.0-fold, at least about 2.5-fold, at least about 5-fold, at least about 10-fold, or more than about 10-fold than said first number.

15. The method of claim 1, further comprising sequencing the amplification products.

16. The method of claim 1, wherein amplifying the first circular polynucleotide comprises extending the first nucleic acid molecule.

17. The method of claim 1, wherein amplifying the first circular polynucleotide comprises hybridizing a first amplification primer to the first circular polynucleotide and extending the first amplification primer.

18. The method of claim 1, wherein amplifying the second circular polynucleotide comprises hybridizing a second amplification primer to the second circular polynucleotide and extending the second amplification primer.

19. The method of claim 1, wherein the second nucleic acid molecule is an RNA molecule or a DNA molecule.

20. The method of claim 1, wherein the cell or tissue is permeabilized and immobilized to a solid support.
